(19) United States
(12) Patent Application Publication
(10) Pub. No.: US 2015/0164831 A1
(43) Pub. Date: Jun. 18, 2015

(54) COLCHICINE FORMULATIONS; METHODS OF MAKING; AND METHODS OF USE THEREOF

(71) Applicant: TAKEDA PHARMACEUTICALS U.S.A., INC., Deerfield, IL (US)

(72) Inventors: Richard H. Roberts, Lakewood, NJ (US); David Erkoboni, Pennington, NJ (US); Bela Kraut, Cherry Hill, NJ (US); Maher Kudsi, Cherry Hill, NJ (US); Gandha V. Naringrekar, Princeton, NJ (US)

(21) Appl. No.: 14/389,523

(22) PCT Filed: Mar. 29, 2013
(86) PCT No.: PCT/US2013/034536

§ 371 Date: Sep. 30, 2014

Related U.S. Application Data
(60) Provisional application No. 61/617,775, filed on Mar. 30, 2012.

(51) Int. Cl. A61K 31/165; A61K 9/20; A61K 9/16

(52) U.S. Cl. CPC A61K 31/165; A61K 9/16; A61K 9/2081

(57) ABSTRACT

Disclosed are new photostable colchicine formulations, methods of preparing the formulations, and uses thereof.

### BACKGROUND

Colchicine, chemical name (−)-N-[(7S,12aS)-1,2,3,10-tetramethoxy-9-oxo-5,6,7,9-tetrahydrobenzo[a]heptalen-7-yl]-acetamide, (N-((7S)-5,6,7,9-tetrahydro-1,2,3,10-tetramethoxy-9-oxobenzo(a)heptalen-7-yl)-acetamide, IUPAC), CAS Registry No. 64-86-8 is a known gout suppressant.

Colchicine is an alkaloid found in extracts of certain plants such as Colchicum autumnale and Gloriosa superba. Colchicine arrests cell division in animals and plants. It has adversely affected spermatogenesis in humans and in some animal species under certain conditions.

Gout (or gouty arthritis) is a disease caused by a build up of uric acid due to an overproduction of uric acid or a reduced ability of the kidney to get rid of uric acid. It is more common in males, postmenopausal women, and people with high blood pressure. Heavy alcohol use, diabetes, obesity, sickle cell anemia, and kidney disease also increase the risk. The condition may also develop in people who take drugs that interfere with uric acid excretion.

Colchicine can be used for treating adults with acute gouty arthritis and pain in attacks of acute gouty arthritis, and also can be used beneficially for treating adults with chronic gout for prophylaxis of acute gout flares. Although its exact mode of action in the relief of gout is not completely understood, colchicine is known to decrease the inflammatory response to urate crystal deposition by inhibiting migration of leukocytes, to interfere with urate deposition by decreasing lactic acid production by leukocytes, to interfere with kinin formation and to diminish phagocytosis and the subsequent anti-inflammatory response. The anti-inflammatory effect of colchicine is relatively selective for acute gouty arthritis. However, other types of arthritis occasionally respond. It is neither an analgesic nor a uricosuric and will not prevent progression to chronic gouty arthritis. It does have a prophylactic, suppressive effect that helps to reduce the incidence of acute attacks and to relieve the residual pain and mild discomfort that patients with gout occasionally experience. In some instances, non-steroidal anti-inflammatory drugs (NSAIDs) may also be prescribed to relieve pain and inflammation in acute gouty arthritis attacks. Strong painkillers, such as codeine, or corticosteroids may also be prescribed to relieve the pain.

Currently a colchicine oral dosage form is commercially available as a purple, film-coated, capsule-shaped tablet (COLCRYS® (colchicine, USP)) containing FD&C blue #2, FD&C red #40, and titanium dioxide.

Certain populations of patients, such as the elderly and pediatric patients, have difficultly swallowing solid, oral dosage forms such as tablets and capsules due to their large size. In such instances, sprinkle forms of an active agent allow for the versatility of administering to different populations of patients.

Colchicine degrades under exposure to light. Beta- and gamma-lumicolchicines are known photoisomers of colchicine (J. Nat Prod. 1988 January-February; 51(1):88-93). Furthermore, colchicine has a bitter taste.

There remains a need in the art for a single, versatile oral colchicine formulation that can be administered to patients that have the ability to swallow traditionally sized dosage forms which at the same time can be administered to patient populations that have difficulty swallowing traditionally sized dosage forms. Such a formulation should exhibit sufficient photostability in traditional oral tablet or capsule form as well as in sprinkle form to minimize the formation of undesirable photodegradants, and should also not degrade or dissolve in a sprinkle administration vehicle (e.g. applesauce) to prevent patient rejection due to poor/bitter taste.

### SUMMARY

In one embodiment, a solid, oral colchicine formulation which can be orally administered as a single unit or as a sprinkle form onto a food vehicle exhibits photostability and optionally taste masking when administered in either form.

In one embodiment, a colchicine dosage form comprises colchicine, a pharmaceutically acceptable excipient, and a light protecting agent selected from a light blocking material, a light absorbing material, or a light blocking material and a light absorbing material; wherein the dosage form can be administered as a sprinkle formulation, wherein the sprinkle formulation does not contain more than 0.06% beta-lumicolchicine and gamma-lumicolchicine combined weight after exposure to 1500 to 3000 lux for 15 minutes (about 2700K or about 6500K color temperature).

In another embodiment, a multiparticulate colchicine dosage form comprises a plurality of coated subunits; wherein each coated subunit comprises a core subunit and a coating surrounding the core subunit, wherein the core subunit comprises colchicine and a pharmaceutically acceptable excipient, wherein the coating comprises a light blocking material, a light absorbing material, or a light blocking material and a light absorbing material; and wherein the dosage form can be administered as a sprinkle formulation, wherein the sprinkle formulation does not contain more than 0.06% beta-lumicolchicine and gamma-lumicolchicine combined weight after exposure to 1500 to 3000 lux for 15 minutes (about 2700K or about 6500K color temperature).

In another embodiment, a method of treating a patient in need of colchicine therapy comprises administering to a patient in need thereof a colchicine dosage form comprising colchicine, a pharmaceutically acceptable excipient, and a light protecting agent selected from a light blocking material, a light absorbing material, or a light blocking material and a light absorbing material; wherein the dosage form can be administered as a sprinkle formulation, wherein the sprinkle formulation does not contain more than 0.06% beta-lumicolchicine and gamma-lumicolchicine combined weight after exposure to 1500 to 3000 lux for 15 minutes (about 2700K or about 6500K color temperature).

In yet another embodiment, a method of treating a patient in need of colchicine therapy comprises administering to a patient in need thereof a multiparticulate colchicine dosage form comprising a plurality of coated subunits; wherein each coated subunit comprises a core subunit and a coating surrounding the core subunit, wherein the core subunit comprises colchicine and a pharmaceutically acceptable excipient, wherein the coating comprises a light blocking material, a light absorbing material, or a light blocking material and a light absorbing material; and wherein the dosage form can be administered as a sprinkle formulation, wherein the sprinkle formulation does not contain more than 0.06% beta-lumicolchicine and gamma-lumicolchicine combined weight after exposure to 1500 to 3000 lux for 15 minutes (about 2700K or about 6500K color temperature).

These and other embodiments, advantages and features of the present invention become clear when detailed description and examples are provided in subsequent sections.

### BRIEF DESCRIPTION OF DRAWINGS

FIG. 1 illustrates a schematic of a crushable colchicine tablet (10) having a tablet matrix (20) and coated subunits (30). The FIGURE is representative only and not to scale.

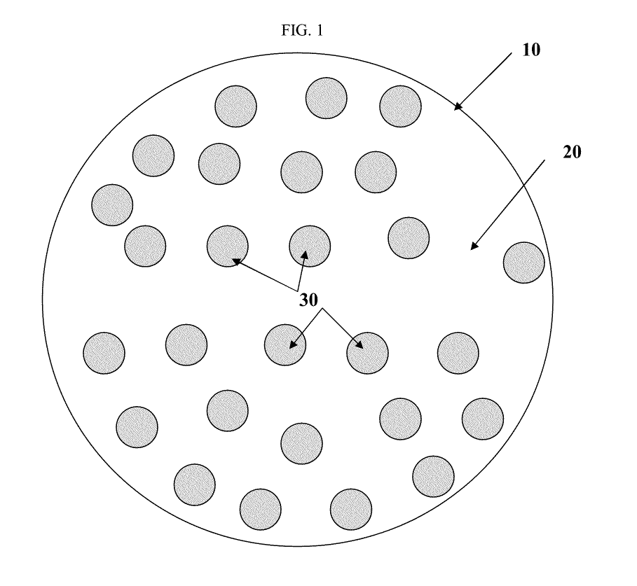

### DETAILED DESCRIPTION

Disclosed herein are solid, oral colchicine formulations which offer the flexibility of either being orally administered as a single unit (e.g., capsule or tablet form ingested whole) or as a sprinkle form onto a food vehicle (either prepared as a sachet or by opening a capsule to release subunits or crushing a tablet to break up a matrix containing subunits) while at the same time exhibiting adequate photostability and optionally taste masking when administered in either form.

The colchicine formulation can be a multiparticulate system containing a plurality of subunits comprising colchicine. “Subunit” includes, but is not limited to, a minitablet, a bead, a spheroid, a microsphere, a seed, a pellet, a caplet, a microcapsule, a granule, a particulate, and the like that can provide an oral dosage form alone or when combined with other subunits.

In one embodiment, the formulation comprises a crushable matrix tablet comprising a plurality of coated colchicine subunits and a pharmaceutically acceptable crushable tablet matrix excipient, wherein each subunit comprises a core subunit comprising colchicine and a pharmaceutically acceptable excipient; and a coating surrounding the core subunit forming a coated subunit. Either one or both of the tablet matrix and the subunit coating further comprises a light blocking material, a light absorbing material, or both a light blocking material and a light absorbing material. The crushable tablet may be prepared by compression or other suitable means. When administered as a sprinkle formulation over food such as applesauce, the crushable tablets can be crushed with a low force (e.g., crushable or broken up with finger pressure such as crushable between finger and thumb with minimal effort) to release the multiparticulate system without damaging the subunits. It has been surprisingly found that the presence of a light blocking material and a light absorbing material in the tablet matrix provides superior photoprotection for the colchicine even in the sprinkle form.

As used herein “crushable tablet” means a tablet that can be size reduced by manual pressure to result in piece sizes that are smaller than half the original tablet size, specifically the size of the subunits used to prepare the crushable tablet, or smaller sizes.

In another embodiment, the formulation comprises a capsule comprising a plurality of subunits, wherein each subunit comprises a core subunit comprising colchicine and a pharmaceutically acceptable excipient; and a coating surrounding the core subunit forming a coated subunit. The coating and optionally the capsule shell comprises a light blocking material, a light absorbing material, or a light blocking material and a light absorbing material. The capsule can be a hard capsule shell. When administered as a sprinkle formulation over food such as applesauce, the capsules can merely be twisted or broken open and the subunits added to the food without breaking the subunits.

The core subunits may be prepared by, for example, dry granulation or wet granulation followed by compression or compaction, melt extrusion, extrusion/spheronization, molding, spheronization, layering (e.g., spray layering suspension or solution), granule formation, and the like. Examples of such techniques include direct compression, using appropriate punches and dies, the punches and dies are fitted to a suitable rotary tableting press; wet granulation using suitable granulating equipment such as a high shear granulator to form wetted particles to be dried into granules or subunits; granulation followed by compression using appropriate punches and dies, the punches and dies are fitted to a suitable rotary tableting press; extrusion of a wet mass to form a cylindrical extrudate to be cut into desire lengths or break into lengths under gravity and attrition; extrusion/spheronization where the extrudate is rounded into spherical particles and densified by spheronization; spray layering of a suspension or solution onto an inert core using a technique such as a convention pan or Wurster column; injection or compression molding using suitable molds fitted to a compression unit; and the like.

In one embodiment, the core subunits are core granules, which can be prepared according to the processes disclosed in U.S. Pat. No. 7,207,505B2 to Bender et al. “Method for producing small granules” the contents of which are incorporated herein.

The size range for the core granules can be selected from within an overall spectrum of possible sizes from about 74 micrometers to about 2000 micrometers (diameter), specifically about 100 to about 1500 micrometers, more specifically about 150 to about 1000 micrometers, and yet more specifically about 200 to about 750 micrometers.

The core granules may with the addition of a lubricant be incorporated directly into a pharmaceutical solid dosage form. Or alternatively, the core granules can be passed through a separate screening machine to further sort out and eliminate granules falling outside of a selected size range. By selectively utilizing one or more screen sizes, the resulting granules can be made highly uniform in size.

In another embodiment, the core granules may be prepared by the process comprising dissolving or suspending a polymeric binder in a liquid to form a granulation liquid; wet granulating the active agent and other pharmaceutically acceptable excipients with the granulation liquid to form a wet granule; drying the wet granule to form a dry granule; and milling the dried granule to form core granules.

As used herein, “pharmaceutically acceptable excipient” means any other component added to the pharmaceutical formulation other than the active agent. Excipients may be added to facilitate manufacture, enhance stability, enhance product characteristics, enhance bioavailability, enhance patient acceptability, etc. Pharmaceutical excipients include carriers, fillers, binders, disintegrants, lubricants, glidants, granulating agent, compression aids, colorants, sweeteners, preservatives, suspending agents, dispersing agents, film formers, flavors, printing inks, buffer agents, pH adjusters, preservatives etc. In some instances, a single material will meet two or more of the foregoing general classifications.

Exemplary pharmaceutically acceptable excipients include fillers, such as a water insoluble filler, water soluble filler, or a combination thereof. The filler may be a water insoluble filler, such as carnauba wax, stearic acid, silicon dioxide, titanium dioxide, talc, alumina, starch, kaolin, polacrilin potassium, powdered cellulose, microcrystalline cellulose, sodium citrate, dicalcium phosphate or a combination thereof. Exemplary water-soluble fillers include water soluble sugars and sugar alcohols, specifically lactose, glucose, fructose, sucrose, mannose, dextrose, galactose, the corresponding sugar alcohols and other sugar alcohols, such as mannitol, sorbitol, xylitol, or a combination thereof.

Exemplary binders include alginic acid, a carbomer, carboxymethylcellulose calcium, carboxymethylcellulose sodium, carrageenan, cellulose acetate phthalate, chitosan, ethyl cellulose, guar gum, hydroxyethyl cellulose, hydroxyethylmethyl cellulose, hydroxypropyl cellulose, hydroxypropyl methyl cellulose, methyl cellulose, microcrystalline cellulose, poloxamer, polyethylene oxide, polymethacrylates, povidone, a saccharide, starch, partially pregelatinized starch, and the like, or a combination thereof.

Exemplary disintegrants include alginic acid, carboxymethylcellulose calcium, carboxymethylcellulose sodium, cross-linked sodium carboxymethylcellulose (sodium croscarmellose), powdered cellulose, chitosan, croscarmellose sodium, crospovidone, guar gum, low substituted hydroxypropyl cellulose, methyl cellulose, microcrystalline cellulose, sodium alginate, sodium starch glycolate, partially pregelatinized starch, pregelatinized starch, starch, sodium carboxymethyl starch, and the like, or a combination thereof.

Exemplary lubricants include calcium stearate, magnesium stearate, glyceryl behenate, glyceryl palmitostearate, hydrogenated castor oil, light mineral oil, sodium lauryl sulfate, magnesium lauryl sulfate, sodium stearyl fumarate, stearic acid, zinc stearate, or a combination thereof.

Exemplary glidants include colloidal silica, amorphous silica, precipitated silica, talc, calcium phosphate tribasic, calcium silicate, magnesium silicate, magnesium trisilicate, and the like, or a combination thereof.

In another embodiment, the core subunits can be prepared by compression into a compressed form (e g, minitablets) using conventional tableting equipment using standard techniques. Techniques and compositions for making tablets (compressed and molded) are described in Remington's Pharmaceutical Sciences, (Aurther Osol., editor), 1553-1593 (1980).

Layering techniques suitable to prepare the core subunits include coating inert cores with a layering solution or dispersion of colchicine and a pharmaceutically acceptable excipient. Repeated layering can be used to build the subunit size and increase active agent amount.

Exemplary liquids that can be used to prepare the layering dispersion or solution for the layering technique include water, lower alkyl alcohols (e.g., methanol, ethanol, n-propanol, isopropanol, etc.), lower alkyl ketones or acetates (e.g., acetone, ethyl acetate, etc.), lower alkyl ethers (e.g., ethyl ether, tetrahydrofuran, etc.), acetonitrile, lower halogenated alkyls (e.g., dichloromethane, etc.), or a combination thereof.

Materials suitable for use as the inert cores upon which layers containing colchicine and a pharmaceutically acceptable excipient are applied onto include pharmaceutically acceptable materials that have appropriate dimensions and firmness. Examples of such materials are polymers e.g. plastic resins; inorganic substances, e.g. silica, glass, hydroxyapatite, salts (sodium or potassium chloride, calcium or magnesium carbonate, mono- di- or tri- calcium phosphate) and the like; organic substances, e.g. activated carbon, acids (citric, fumaric, tartaric, ascorbic and the like acids), and saccharides and derivatives thereof. The saccharides include sugars, oligosaccharides, polysaccharides and their derivatives, for example, glucose, rhamnose, galactose, lactose, sucrose, mannitol, sorbitol, dextrin, maltodextrin, cellulose, microcrystalline cellulose, sodium carboxymethyl cellulose, starches (maize, rice, potato, wheat, tapioca) and the like. Waxes including as examples carnauba, candelilla, white and microcrystalline.

The inert core can have an average diameter of about 50 to about 2500 micrometers, specifically about 100 to about 2000 micrometers, yet more specifically about 250 to about 1500 micrometers, and still yet more specifically about 500 to about 1000 micrometers.

Depending upon the process used to prepare the core subunits, the size of the core subunit can be varied to a targeted range. In one embodiment, the core subunits have an average diameter of about 74 to about 4000 micrometers, specifically about 500 to about 3500 micrometers, yet more specifically about 1000 to about 3000 micrometers, more specifically about 1500 to about 2750 micrometers.

The core granules also have a narrow particle size distribution wherein 0 to about 30 weight percent (wt %) of the core granules have a particle size within plus or minus 75 wt % of the mean particle size (e.g. nₗₑₛₛ=wt % of granules having a size less than 75 wt % of the mean size and n_{greater}=wt % of granules having a size greater than 75 wt % of the mean size where nₗₑₛₛ+n_{greater} does not exceed about 30 wt %). Specifically 0 to about 20 wt % of the core granules have a particle size within plus or minus 75 wt % of the mean particle size. More specifically 0 to about 10 wt % of the core granules have a particle size within plus or minus 75 wt % of the mean particle size.

In one embodiment, the core subunits are minitablets having an average length of its longest dimension of about 500 to about 4000 micrometers, specifically about 750 to about 3500 micrometers, yet more specifically about 1000 to about 3000 micrometers, more specifically about 1250 to about 2500 micrometers, still yet more specifically about 1500 to about 2250 micrometers, and more specifically about 1750 to about 2000 micrometers.

Each subunit can contain any amount of colchicine up to about 99 wt %, specifically about 0.05 to about 60 wt %, more specifically about 0.10 to about 30 wt %, yet more specifically about 0.15 to about 15 wt %, and still yet more specifically about 0.20 to about 5 wt % based on the total weight of the uncoated subunit.

In one embodiment, the amount of colchicine per subunit is about 0.001 to about 0.1 mg, specifically about 0.005 to about 0.01 mg.

The core subunits described herein are “coated” to result in coated subunits. The core subunits can be coated with a functional or non-functional coating, or a combination of functional and non-functional coatings. By “functional coating” is meant to include a coating that modifies the release properties of the total composition, for example, a sustained-release or delayed-release coating. By “non-functional coating” is meant to include a coating that is not a functional coating, for example, a cosmetic coating. A non-functional coating can have some impact on the release of the active agent due to the initial dissolution, hydration, perforation of the coating, etc., but would not be considered to be a significant deviation from the non-coated composition. A non-functional coating can also mask the taste of the granule composition including the active pharmaceutical ingredient. As discussed above, the coating comprises a light blocking material, a light absorbing material, or a light blocking material and a light absorbing material.

The weight gain of the coating can be in an amount of about 0.1 to about 100% weight gain based on the weight of the core subunit, specifically about 2 to about 75%, more specifically about 4 to about 50%, and yet more specifically about 6 to about 25% weight gain based on the total weight of the core subunit and coating material; which in some instances can result in a total percent coating amount of about 0.1 to about 50%, specifically about 1 to about 43%, more specifically about 3 to about 34% and yet more specifically about 5 to about 20%. The amounts can be greater or lesser depending upon the composition of the core subunit, size of the core subunit, amount of plasticizer or surfactant, among other things.

Suitable polymeric coating material for use to prepare the coated subunits includes a water soluble polymer that is a film forming polymer. For example, the water soluble film forming polymers can be selected from the group comprising hydroxyalkylcelluloses such as hydroxymethylcellulose, hydroxyethylcellulose, hydroxypropylcellulose and hydroxybutylcellulose; hydroxyalkyl alkylcelluloses such as hydroxyethyl methylcellulose and hydroxypropyl methylcellulose; carboxyalkylcelluloses such as carboxymethylcellulose; alkali metal salts of carboxyalkylcelluloses such as sodium carboxymethylcellulose; carboxyalkyl alkylcelluloses such as carboxymethyl ethylcellulose; carboxyalkylcellulose esters; starches; pectines such as sodium carboxymethylamylopectine; chitin derivates such as chitosan; polysaccharides such as alginic acid, alkali metal and ammonium salts thereof, carrageenans, galactomannans, traganth, agar-agar, gum arabicum, guar gum and xanthan gum; polyacrylic acids and the salts thereof; polymethacrylic acids and the salts thereof; methacrylate copolymers; polyvinylalcohol; polyvinylpyrrolidone; copolymers of polyvinylpyrrolidone with vinyl acetate; polyalkylene oxides such as polyethylene oxide and polypropylene oxide and copolymers of ethylene oxide and propylene oxide; or a combination thereof. Other pharmaceutically acceptable polymers that exhibit similar as defined above physico-chemical properties as defined above are equally suitable.

Specific water soluble film forming polymers are for example hydroxypropyl methylcellulose, polymethacrylate, hydroxypropylcellulose, polyvinyl alcohol, or a polyvinylpyrrolidone; more specifically a hydroxypropyl methylcellulose (HPMC) or polyvinyl alcohol. HPMC contains sufficient hydroxypropyl and methoxy groups to render it water-soluble. HPMC having a methoxy degree of substitution from about 0.8 to about 2.5 and a hydroxypropyl molar substitution from about 0.05 to about 3.0 are generally water-soluble. Methoxy degree of substitution refers to the average number of methyl ether groups present per anhydroglucose unit of the cellulose molecule. Hydroxypropyl molar substitution refers to the average number of moles of propylene oxide which have reacted with each anhydroglucose unit of the cellulose molecule. Suitable HPMC include those having a viscosity from about 1 to about 100 mPa·s, specifically about 3 to about 15 mPa·s, and more specifically about 5 mPa·s. Polyvinyl alcohol is partially hydrolyzed and retains some of the residual acetate groups to insure its solubility in water at room temperature.

Other coating materials, which may be suitable for functional coatings include acrylic polymers, alkylcelluloses, shellac, zein, hydrogenated vegetable oil, hydrogenated castor oil, vinyl acetate, vinyl acetate copolymers, polyethylene oxide, or a combination thereof.

Suitable acrylic polymers include, for example, acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly(methacrylic acid anhydride), methyl methacrylate, polymethacrylate, poly(methyl methacrylate) copolymer, polyacrylamide, aminoalkyl methacrylate copolymer, glycidyl methacrylate copolymers, or a combination thereof. The acrylic polymer may comprise methacrylate copolymers described in NF XXIV as fully polymerized copolymers of acrylic and methacrylic acid esters with a low content of quaternary ammonium groups.

Exemplary polymethacrylates include copolymers of acrylic and methacrylic acid esters, such as a. an aminomethacrylate copolymer USP/NF such as a poly(butyl methacrylate, (2-dimethyl aminoethyl)methacrylate, methyl methacrylate) 1:2:1 (e.g., EUDRAGIT E 100, EUDRAGIT EPO, and EUDRAGIT E 12.5; CAS No. 24938-16-7); b. a poly(methacrylic acid, ethyl acrylate) 1:1 (e.g., EUDRAGIT L30 D-55, EUDRAGIT L100-55, EASTACRYL 30D, KOLLICOAT MAE 30D AND 30DP; CAS No. 25212-88-8); c. a poly(methacrylic acid, methyl methacrylate) 1:1 (e.g., EUDRAGIT L 100, EUDRAGIT L 12.5 and 12.5 P; also known as methacrylic acid copolymer, type A NF; CAS No. 25806-15-1); d. a poly(methacrylic acid, methyl methacrylate) 1:2 (e.g. EUDRAGIT S 100, EUDRAGIT S 12.5 and 12.5P; CAS No. 25086-15-1); e. a poly(methyl acrylate, methyl methacrylate, methacrylic acid) 7:3:1 (e.g. Eudragit FS 30 D; CAS No. 26936-24-3); f a poly(ethyl acrylate, methylmethacrylate, trimethylammonioethyl methacrylate chloride) 1:2:0.2 or 1:2:0.1 (e.g., EUDRAGITS RL 100, RL PO, RL 30 D, RL 12.5, RS 100, RS PO, RS 30 D, or RS 12.5; CAS No. 33434-24-1); g. a poly(ethyl acrylate, methyl methacrylate) 2:1 (e.g. EUDRAGIT NE 30 D, Eudragit NE 40D, Eudragit NM 30D; CAS No. 9010-88-2); and the like, or a combination thereof.

Suitable alkylcelluloses include, for example, methylcellulose, ethylcellulose, and the like, or a combination thereof. Exemplary water based ethylcellulose coatings include AQUACOAT, a 30% dispersion further containing sodium lauryl sulfate and cetyl alcohol, available from FMC, Philadelphia, Pa.; SURELEASE a 25% dispersion further containing a stabilizer or other coating component (e.g., ammonium oleate, dibutyl sebacate, colloidal anhydrous silica, medium chain triglycerides, etc.) available from Colorcon, West Point, Pa.; ethyl cellulose available from Aqualon or Dow Chemical Co (Ethocel), Midland, Mich. Those skilled in the art will appreciate that other cellulosic polymers, including other alkyl cellulosic polymers, can be substituted for part or all of the ethylcellulose.

Other suitable materials that can be used to prepare a functional coating include hydroxypropyl methylcellulose acetate succinate (HPMCAS); cellulose acetate phthalate (CAP); a polyvinylacetate phthalate; neutral or synthetic waxes, fatty alcohols (such as lauryl, myristyl, stearyl, cetyl or specifically cetostearyl alcohol), fatty acids, including fatty acid esters, fatty acid glycerides (mono-, di-, and tri-glycerides), hydrogenated fats, hydrocarbons, normal waxes, stearic acid, stearyl alcohol, hydrophobic and hydrophilic materials having hydrocarbon backbones, or a combination thereof. Suitable waxes include beeswax, glycowax, castor wax, carnauba wax, microcrystalline wax, candelilla, and wax-like substances, e.g., material normally solid at room temperature and having a melting point of from about 30° C. to about 100° C., or a combination thereof.

In other embodiments, the functional coating may comprise digestible, long chain (e.g., C₈-C₅₀, specifically C₁₂-C₄₀), substituted or unsubstituted hydrocarbons, such as fatty acids, fatty alcohols, glyceryl esters of fatty acids, mineral and vegetable oils, waxes, or a combination thereof. Hydrocarbons having a melting point of between about 25° C. and about 90° C. may be used. Specifically, long chain hydrocarbon materials, fatty (aliphatic) alcohols can be used.

The coatings can optionally contain additional pharmaceutically acceptable excipients such as a plasticizer, a stabilizer, a water-soluble component (e.g. pore formers), an anti-tacking agent (e.g., talc), a surfactant, and the like, or a combination thereof.

The functional coating may comprise a release-modifying agent, which affects the release properties of the functional coating. The release-modifying agent can, for example, function as a pore-former or a matrix disrupter. The release-modifying agent can be organic or inorganic, and include materials that can be dissolved, extracted or leached from the coating in the environment of use. The release-modifying agent can comprise one or more hydrophilic polymers including cellulose ethers and other cellulosics, such as hydroxypropyl methylcellulose, hydroxypropylcellulose, hydroxyethylcellulose, methyl cellulose, cellulose acetate phthalate, or hydroxypropyl methylcellulose acetate phthalate; povidone; polyvinyl alcohol; an acrylic polymer, such as gastric soluble Eudragit FS 30D, pH sensitive Eudragit L30D 55, L 100, S 100, or L 100-55; or a combination thereof. Other exemplary release-modifying agents include a povidone; a saccharide (e.g., lactose, and the like); a metal stearate; an inorganic salt (e.g., dibasic calcium phosphate, sodium chloride, and the like); a polyethylene glycol (e.g., polyethylene glycol (PEG) 1450, and the like); a sugar alcohol (e.g., sorbitol, mannitol, and the like); an alkali alkyl sulfate (e.g., sodium lauryl sulfate); a polyoxyethylene sorbitan fatty acid ester (e.g., polysorbate); or a combination thereof. Exemplary matrix disrupters include water insoluble organic or inorganic material. Organic polymers including but not limited to cellulose, cellulose ethers such as ethylcellulose, cellulose esters such as cellulose acetate, cellulose acetate butyrate and cellulose acetate propionate; and starch can function as matrix disrupters. Examples or inorganic disrupters include many calcium salts such as mono-, di- and tri calcium phosphate; silica and, talc.

The coating may optionally contain a plasticizer to improve the physical properties of the coating. For example, because ethylcellulose has a relatively high glass transition temperature and does not form flexible films under normal coating conditions, it may be advantageous to add plasticizer to the ethylcellulose before using the same as a coating material. Generally, the amount of plasticizer included in a coating solution is based on the concentration of the polymer, e.g., can be from about 1% to about 200% depending on the polymer but is most often from about 1 wt % to about 100 wt % of the polymer. Concentrations of the plasticizer, however, can be determined by routine experimentation.

Examples of plasticizers for ethylcellulose and other celluloses include plasticizers such as dibutyl sebacate, diethyl phthalate, triethyl citrate, tributyl citrate, triacetin, or a combination thereof, although it is possible that other water-insoluble plasticizers (such as acetylated monoglycerides, phthalate esters, castor oil, etc.) can be used.

Examples of plasticizers for acrylic polymers include citric acid esters such as triethyl citrate NF, tributyl citrate, dibutyl phthalate, 1,2-propylene glycol, polyethylene glycols, propylene glycol, diethyl phthalate, castor oil, triacetin, or a combination thereof, although it is possible that other plasticizers (such as acetylated monoglycerides, phthalate esters, castor oil, etc.) can be used.

Suitable methods can be used to apply the coating material to the surface of the subunits. Processes such as simple or complex coacervation, interfacial polymerization, liquid drying, thermal and ionic gelation, spray drying, spray chilling, fluidized bed coating, pan coating, or electrostatic deposition may be used.

In certain embodiments, an optional intermediate coating is used between the core subunit and an exterior coating. Such an intermediate coating can be used to protect the active agent or other component of the core subunit from the material used in the exterior coating or to provide other properties. Exemplary intermediate coatings typically include water-soluble film forming polymers. Such intermediate coatings may include film forming polymers such as hydroxyethyl cellulose, hydroxypropyl cellulose, gelatin, hydroxypropyl methylcellulose, polyethylene glycol, polyethylene oxide, and the like, or a combination thereof; and a plasticizer. Plasticizers can be used to reduce brittleness and increase tensile strength and elasticity. Exemplary plasticizers include polyethylene glycol propylene glycol and glycerin.

In one embodiment, the plurality of coated subunits or uncoated subunits of the multiparticulate system can be loaded into hard capsule shells (e.g., gelatin capsules) using techniques well-known in the art.

In another embodiment, the plurality of coated subunits or uncoated subunits of the multiparticulate system is prepared as a sachet using techniques well-known in the art.

In still yet another embodiment, the plurality of coated subunits or uncoated subunits of the multiparticulate system can be mixed with an appropriate excipient and compressed into a crushable tablet. The tablet can either be administered whole or lightly crushed, such as with finger pressure, to release the individual subunits and sprinkled over an appropriate vehicle (e.g., applesauce). The crushable tablet can be prepared by dry blending excipient with the coated subunits and compressed using techniques know in the art with care taken in the process to avoid damaging the individual subunits. In another embodiment the coated subunits can be granulated with the excipients in a fluid bed and then compressed using techniques known in the art with care taken in the process to avoid damaging the individual subunits. Suitable excipients to prepare the crushable tablet include those typically used for chewable tablets including mono- and di-saccharides, sugar polyols, and the like, or a combination thereof; those that are highly compactable such as microcrystalline cellulose and modified starches or a combination thereof; those that are easily deformed such as waxes such as carnauba, white and microcrystalline and wax like materials such as stearic acid, cetyl alcohol, polyethylene glycol, and the like. Exemplary mono- and di-saccharide, sugar polyol excipients include mannitol, sorbitol, xylitol, maltitol, lactose, sucrose, maltose, or a combination thereof. Optional pharmaceutical excipients such as diluents, lubricants, glidants, flavorants, colorants, etc. or a combination thereof may also be included in the compression matrix.

The hardness of the crushable tablet can vary significantly depending on the size and geometry of the tablet. Crushable tablet hardness can vary from about 0.25 to about greater than 30 kiloponds (Kp) depending on the size and geometry, specifically about 1 to about 25 Kp, more specifically about 2 to about 20 Kp. In one embodiment, the hardness of the crushable tablet can be about 0.5 to about 3.7 kiloponds (Kp), specifically about 1.2 to about 3 Kp, and more specifically about 1.9 to about 2.3 Kp. Crushable tablet hardness can be measured using a Schleuniger hardness tester Model 8M. The speed of travel for the jaws of a hardness tester can significantly influence the values generated during testing. The rate of travel for this model is programmable and the range is about 0.1 to about 5 mm per second.

The friability of the crushable tablet can be about 0.05 to about 2.0%, specifically about 0.3 to about 1.0%, and more specifically about 0.4 to about 0.6%. Friability can be measured using a Roche type friabilator at 100 revolutions over a period of 4 minutes. In several embodiments, the friability is not more than 0.8%.

The tensile strength of the crushable tablet can be about 10 kpascal to about 8000 kpascal, specifically about 30 kPascal to about 6000 kPascal, and more specifically about 50 kPascal to about 4000 kPascal. The tensile strength can be determined by measuring the tablet hardness in kiloponds using a Schleuniger 8M hardness tester and converting it to tensile strength using the following equation: TS=10F/πd² (2.84 t/d−0.126 t/c₁+3.15 c₁/d+0.001) where TS=Tensile strength, F=Breaking force (hardness), d=Tablet diameter, t=Tablet overall thickness and c₁=Tablet belly band length.

In one embodiment, the solid, oral colchicine formulation is a scored tablet meeting FDA guidelines and criteria, for example in the draft guidance “Guidance for Industry Tablet Scoring: Nomenclature, Labeling, and Data for Evaluation” available from the U.S. Department of Health and Human Services (DHHS), Food and Drug Administration (FDA), Center for Drug Evaluation and Research (CDER) August 2011, which is incorporated herein in its entirety. Exemplary guidelines and criteria are: the dosage amount meant to be achieved after splitting the tablet should not be below the minimum therapeutic dose indicated on the approved labeling, the scored dosage form should be safe to handle and not pose risk of unintended drug exposure, and the split tablet portions should meet the same finished-product testing requirements as for a whole-tablet product with equivalent strength.

In one embodiment, the crushable colchicine tablet meets the foregoing FDA criteria for Tablet Scoring and has a functional score.

The crushable tablet matrix and the coating of the coated subunits comprise a light blocking material (e.g. an opacifier), a light absorbing material (e.g. a colorant), or a combination of a light blocking material and a light absorbing material.

Exemplary pharmaceutically acceptable opacifiers include pigments such as titanium dioxide, an iron oxide (e.g., Fe₂O₃, Fe₂O₃.H₂O, FeO.Fe₂O₃), zinc oxide, aluminum oxide, and certain aluminum lakes. Other opacifiers include clays such as kaolin, bentonite, and the like; and insoluble inorganic salts such as calcium carbonate, calcium phosphate.

The total amount of light blocking material of the coating can be 0 to about 50 wt % based on the total weight of the coating, specifically about 0.01 to about 30, more specifically about 0.5 to about 20, and yet more specifically about 1 to aobut 15 wt %.

The total amount of light blocking material of the crushable tablet matrix can be about 0.1 to about 25 wt % based on the total weight of the tablet, specifically about 1 to about 15, more specifically about 2 to about 10 wt %, and yet more specifically about 3 to about 7 wt % based on the total weight of the tablet.

Exemplary colorants for use in the coatings and crushable tablet matrix include any U.S. Food and Drug Administration approved colorants for oral use including the FD&C and D&C colors and lakes, such as FD&C Red No. 40 Aluminum Lake; FD&C Red No. 4 Lake; D&C Red No. 6 Lake; D&C Red No. 7 Lake; D&C Red No. 17 Lake; D&C Red No. 21 Lake; D&C Red No. 22 Lake; D&C Red No. 27 Lake; D&C Red No. 28 Lake; D&C Red No. 30 Lake; D&C Red No. 31 Lake; D&C Red No. 33 Lake; D&C Red No. 34 Lake; D&C Red No. 36 Lake; D&C Violet No. 2 Lake; D&C Yellow No. 10 Aluminum Lake; FD&C Yellow No. 6 Aluminum Lake; FD&C Yellow No. 5 Lake; D&C Yellow No. 7 Lake; D&C Yellow No. 8 Lake; D&C FD&C Blue No. 1 Lake; FD&C Blue No. 2 Aluminum Lake; D&C Blue No. 4 Lake; FD&C Green No. 3 Lake; D&C Green No. 5 Lake; D&C Green No. 6 Lake; D&C Orange No. 4 Lake; D&C Orange No. 5 Lake; D&C Orange No. 10 Lake; D&C Orange No. 11 Lake; FD&C Red No. 40; FD&C Red No. 4; D&C Red No. 6; D&C Red No. 7; D&C Red No. 17; D&C Red No. 21; D&C Red No. 22; D&C Red No. 27; D&C Red No. 28; D&C Red No. 30; D&C Red No. 31; D&C Red No. 33; D&C Red No. 34; D&C Red No. 36; D&C Red No. 39; D&C Violet No. 2; FD&C Yellow No. 6; FD&C Yellow No. 5; D&C Yellow No. 7; D&C Yellow No. 8; D&C Yellow No. 10; D&C Yellow No. 11; FD&C Blue No. 1; FD&C Blue No. 2; D&C Blue No. 4; D&C Blue No. 9; FD&C Green No. 3; D&C Green No. 5; D&C Green No. 6; D&C Green No. 8; D&C Orange No. 4; D&C Orange No. 5; D&C Orange No. 10; D&C Orange No. 11; or a combination thereof.

In one embodiment, the colorant is a combination of FD&C Red No. 40 aluminum lake, FD&C Yellow No. 6 aluminum lake, and FD&C Blue No. 1 aluminum lake, and the opacifier is titanium dioxide. In another embodiment, the colorant is D&C Yellow No. 10 aluminum lake and the opacifier is titanium dioxide. In yet another embodiment, the colorant is FD&C Blue No. 2 aluminum lake and the opacifier is titanium dioxide. In still another embodiment, the colorant is a combination of FD&C Red No. 40 aluminum lake and FD&C Blue No. 2 aluminum lake, and the opacifier is titanium dioxide. In still another embodiment, the colorant is a combination of FD&C Red No. 40 aluminum lake and D&C Yellow No. 10 aluminum lake, and the opacifier is titanium dioxide. In yet another embodiment, the colorant is a combination of FD&C Red No. 40 aluminum lake, FD&C Yellow No. 6 aluminum lake, D&C Yellow No. 10 aluminum lake, and FD&C Blue No. 2 aluminum lake and the opacifier is titanium dioxide.

The dye strength of any one of the dyes can be about 2 to about 50, specifically about 3 to about 45, more specifically about 5 to about 35, and yet more specifically about 5 to about 17.

The total amount of light absorbing material of the coating can be about 0.01 to about 50 wt % based on the total coating weight, specifically about 1 to about 40 wt %, and more specifically about 5 to about 30 wt %.

The total amount of light absorbing material of the crushable tablet matrix can be about 0.01 to about 15 wt % based on the total weight of the tablet, specifically about 0.05 to about 10, more specifically about 0.1 to about 5 wt %, and yet more specifically about 0.2 to about 2.5 wt % based on the total weight of the tablet.

In one embodiment, the coating, crushable tablet matrix, or coating and crushable tablet matrix contain a blend of an Aluminum lake and opacifier, specifically titanium dioxide.

The solid, oral colchicine formulations can be formulated for immediate-, sustained-, extended-, delayed- or pulsed-release profiles both in vivo and in vitro. An immediate-release formulation is one that has not been modified to provide a release profile that is delayed, extended, sustained, pulsed, or controlled. By “immediate-release” is meant a conventional or non-modified release. As used herein, immediate-release is not controlled-, sustained-, extended-, delayed- or pulsed-release.

The solid, oral colchicine formulation can be described by its pharmacokinetic or dissolution profiles. “Pharmacokinetic parameters” describe the in vivo characteristics of an active agent (or surrogate marker for the active agent) over time, such as plasma concentration (C), Cₘₐₓ, Cₙ, C₂₄, Tₘₐₓ, and AUC. “Cₘₐₓ” is the measured concentration of the active agent in the plasma at the point of maximum concentration. “Cₙ” is the measured concentration of an active agent in the plasma at about n hours after administration. “C₂₄” is the measured concentration of an active agent in the plasma at about 24 hours after administration. The term “Tₘₐₓ” refers to the time at which the measured concentration of an active agent in the plasma is the highest after administration of the active agent. “AUC” is the area under the curve of a graph of the measured concentration of an active agent (typically plasma concentration) vs. time, measured from one time point to another time point. For example AUC₀₋ₜ is the area under the curve of plasma concentration versus time from time 0 to time t. The AUC_{0-∞} or AUC_{0-INF} is the calculated area under the curve of plasma concentration versus time from time 0 to time infinity.

“Bioavailability” means the extent or rate at which an active agent is absorbed into a living system or is made available at the site of physiological activity. For active agents that are intended to be absorbed into the bloodstream, bioavailability data for a given formulation may provide an estimate of the relative fraction of the administered dose that is absorbed into the systemic circulation. “Bioavailability” can be characterized by one or more pharmacokinetic parameters.

In one embodiment, the solid, oral colchicine formulation is bioequivalent to a reference drug. In one embodiment, bioequivalence is any definition thereof as promulgated by the U.S. Food and Drug Administration or any successor agency thereof. In a specific embodiment, bioequivalence is determined according to the Federal Drug Administration's (FDA) guidelines and criteria, including “GUIDANCE FOR INDUSTRY BIOAVAILABILITY AND BIOEQUVALENCE STUDIES FOR ORALLY ADMINISTERED DRUG PRODUCTS—GENERAL CONSIDERATIONS” available from the U.S. Department of Health and Human Services (DHHS), Food and Drug Administration (FDA), Center for Drug Evaluation and Research (CDER) March 2003 Revision 1; and “GUIDANCE FOR INDUSTRY STATISTICAL APPROACHES TO ESTABLISHING BIOEQUIVALENCE” DHHS, FDA, CDER, January 2001, both of which are incorporated herein in their entirety.

In another embodiment, bioequivalence is determined according to the European Medicines Agency (EMEA) document “Note for Guidance on the Investigation of Bioavailability and Bioequivalence”, issued Jul. 26, 2001, available from EMEA.

“Reference drug” means the oral colchicine tablet product as described in U.S. Federal Food and Drug Administration's New Drug Application No. 022352 approved on Jul. 29, 2009 (0.6 mg colchicine) and by its brand name Colcrys®. Colcrys® tablets are supplied for oral administration as purple, film-coated, capsule-shaped tablets (0.1575″×0.3030″) containing 0.6 mg of the active ingredient colchicine USP; inactive ingredients: carnauba wax, FD&C blue #2, FD&C red #40, hypromellose, lactose monohydrate, magnesium stearate, microcrystalline cellulose, polydextrose, polyethylene glycol, pregelatinized starch, sodium starch glycolate, titanium dioxide, and triacetin. Colcrys® is formulated for immediate-release.

In one embodiment, the colchicine formulation is bioequivalent to a reference drug according to New Drug Application No. 022352 (Colcrys® 0.6 mg) when tested in a group of five or more healthy humans in the fasted or fed state.

In an embodiment, bioequivalence of the colchicine formulation to a reference drug is determined by an in vivo bioequivalence study to determine a pharmacokinetic parameter for the colchicine formulation. Specifically, bioequivalence can be determined by an in vivo bioequivalence study comparing a pharmacokinetic parameter for the two compositions. A pharmacokinetic parameter for the colchicine formulation or the reference drug can be measured in a single or multiple dose bioequivalence study using a replicate or a nonreplicate design. For example, the pharmacokinetic parameters for a colchicine formulation of the present invention and for a reference drug can be measured in a single dose bioequivalence study using a two-period, two-sequence crossover design. Alternately, a four-period, replicate design crossover study may also be used. Single doses of the test colchicine formulation and reference drug are administered and blood or plasma levels of the active agent are measured over time. Pharmacokinetic parameters characterizing rate and extent of active agent absorption are evaluated statistically.

The area under the plasma concentration-time curve from time zero to the time of measurement of the last quantifiable concentration (AUC₀₋ₜ) and to infinity (AUC_{0-∞}), Cₘₐₓ, and Tₘₐₓ can be determined according to standard techniques. Statistical analysis of pharmacokinetic data is performed on logarithmic transformed data (e.g., AUC₀₋ₜ, AUC_{0-∞}, or Cₘₐₓ data) using analysis of variance (ANOVA).

In some embodiments a single dose pharmacokinetic study is performed under non-fasted (“fed”) or fasted conditions. When tested under fed conditions, the formulation is administered with a high fat meal. An exemplary high fat meal includes the test meal disclosed in the document Guidance for Industry, Food-Effect Bioavailability and Fed Bioequivalence Studies, U.S. Department of Health and Human Services Food and Drug Administration, Center for Drug Evaluation and Research (CDER), Center for Biologics Evaluation and Research (CBER) issued December 2002 and available at http://www.fda.gov/cder/guidance/index.htm. The exemplary high-fat meal contains approximately 50 percent of the total caloric content of the meal as fat and contains approximately 800 to 1000 calories; 500-600 calories from fat. As used herein, the term “fat” is used in its conventional, art-recognized meaning.

Under U.S. FDA guidelines, two products (e.g. an inventive formulation and Colcrys®) or methods (e.g., dosing under fed versus fasted conditions) are bioequivalent if the 90% Confidence Interval (CI) limits for a ratio of the geometric mean of logarithmic transformed AUC_{0-∞}, AUC₀₋ₜ, and Cₘₐₓ for the two products or two methods are about 0.80 to about 1.25.

To show bioequivalence between two products or methods pursuant to Europe's EMEA guidelines, the 90% CI limits for a ratio of the geometric mean of logarithmic transformed AUC_{0-∞} and AUC₀₋ₜ for the two products or methods are about 0.80 to about 1.25. The 90% CI limits for a ratio of the geometric mean of logarithmic transformed Cₘₐₓ for the two products or methods can have a wider acceptance range when justified by safety and efficacy considerations. For example the acceptance range can be about 0.70 to about 1.43, specifically about 0.75 to about 1.33, and more specifically about 0.80 to about 1.25.

In one embodiment, in a given experiment, a colchicine formulation is considered to be bioequivalent to Colcrys® if both the Test/Reference ratio for the geometric mean of logarithmic transformed AUC_{0-∞}, AUC₀₋ₜ, or Cₘₐₓ ratio along with its corresponding lower and upper 90% CI limits are within a lower limit of about 0.80 and an upper limit of about 1.25. Thus, for direct comparison between a colchicine formulation and Colcrys®, it is sometimes preferred to determine the pharmacokinetic parameters for the colchicine formulation and Colcrys® side-by-side in the same pharmacokinetic study.

In another embodiment, the 90% confidence limits of a ratio of a geometric mean of logarithmic transformed AUC_{0-∞} of the colchicine formulation to a geometric mean of logarithmic transformed AUC_{0-∞} of a reference drug according to New Drug Application No. 022352 is about 0.80 to about 1.25 when tested in a group of five or more healthy humans in the fasted or fed state.

In yet another embodiment, the 90% confidence limits of a ratio of a geometric mean of logarithmic transformed AUC₀₋ₜ of the colchicine formulation to a geometric mean of logarithmic transformed AUC₀₋ₜ of a reference drug according to New Drug Application No. 022352 is about 0.80 to about 1.25 when tested in a group of five or more healthy humans in the fasted or fed state.

In yet another embodiment, the 90% confidence limits of a ratio of a geometric mean of logarithmic transformed Cₘₐₓ of the colchicine formulation to a geometric mean of logarithmic transformed Cₘₐₓ of a reference drug according to New Drug Application No. 022352 is about 0.80 to about 1.25 when tested in a group of five or more healthy humans in the fasted or fed state.

In one embodiment, the formulation is bioequivalent to a reference drug product according to New Drug Application No. 022352 when tested in a group of five or more healthy humans in the fasted or fed state, wherein bioequivalence is determined according to “GUIDANCE FOR INDUSTRY BIOAVAILABILITY AND BIOEQUVALENCE STUDIES FOR ORALLY ADMINISTERED DRUG PRODUCTS—GENERAL CONSIDERATIONS” DHHS, FDA, CDER, March 2003 Revision 1; and “GUIDANCE FOR INDUSTRY STATISTICAL APPROACHES TO ESTABLISHING BIOEQUIVALENCE” DHHS, FDA, CDER, January 2001.

In one embodiment, the formulation has no food effect or substantially no food effect such that a patient has the convenience of taking the formulation with or without food.

“Food” typically means a solid food or mixed solid/liquid food with sufficient bulk and fat content that it is not rapidly dissolved and absorbed in the stomach. In one embodiment, food means a meal, such as breakfast, lunch or dinner. The terms “taken with food”, “fed” and “non-fasted” are equivalent and are as given by FDA guidelines and criteria. In one embodiment, with food means that the dosage form is administered to a patient between about 30 minutes prior to about 2 hours after eating a meal. In another embodiment, with food means that the dosage form is administered at substantially the same time as the eating the meal.

The terms “without food”, “fasted” and “an empty stomach” are equivalent and are as given by FDA guidelines and criteria. In one embodiment, fasted is means the condition wherein no food is consumed within 1 hour prior to administration of the dosage form or 2 hours after administration of the dosage form. In another embodiment, fasted means the condition wherein no food is consumed within 1 hour prior to administration of the dosage form to 2 hours after administration of the dosage form.

“Substantially no food effect” means that the pharmacokinetics are substantially the same for the oral administration of the formulation under fed conditions (“non-fasting”) when compared to administration under fasting conditions. For example, the comparison between Cmax or AUC of a single administration of a formulation under fed conditions to a single administration of the same formulation under fasted conditions results in a percent ratio of Cmax or AUC having a 90% confidence interval upper limit of less than or equal to 125% or a lower limit of greater than or equal to 80%. Such information can be based on logarithmic transformed data. Exemplary study considerations can be found in the Federal Drug Administration's (FDA) guidelines and criteria, including “Guidance for Industry, Food-Effect Bioavailability and Fed Bioequivalence Studies” available from the U.S. Department of Health and Human Services (DHHS), Food and Drug Administration (FDA), Center for Drug Evaluation and Research (CDER) December 2002, incorporated herein in its entirety.

In another embodiment, the 90% confidence limits of a ratio of a geometric mean of logarithmic transformed AUC_{0-∞}, AUC₀₋ₜ, or Cₘₐₓ of the colchicine formulation when tested in a group of five or more healthy humans in the fed state to a geometric mean of logarithmic transformed AUC_{0-∞}, AUC₀₋ₜ, or Cₘₐₓ of the colchicine formulation when tested in a group of five or more healthy humans in the fasted state is about 0.80 to about 1.25.

In one embodiment, the colchicine dosage form exhibits an immediate-release profile in vivo where the Tₘₐₓ is about 4 hours or less, specifically about 3 hours or less, and more specifically about 2 hours or less. The Tₘₐₓ can be determined after administration to a test group of about thirteen or more healthy humans in the fasted state.

The release of colchicine from the colchicine formulation can be described by its dissolution profile. A dissolution profile is a plot of the cumulative amount of active agent released as a function of time. A dissolution profile can be measured utilizing the Drug Release Test <724>, which incorporates standard test USP 32 (Test <711>). A profile is characterized by the test conditions selected such as, for example, apparatus type, shaft speed, temperature, volume, and pH of the dissolution medium. More than one dissolution profile may be measured. For example, a first dissolution profile can be measured at a pH level approximating that of the stomach, and a second dissolution profile can be measured at a pH level approximating that of one point in the intestine or several pH levels approximating multiple points in the intestine.

A highly acidic pH may be employed to simulate the stomach and a less acidic to basic pH may be employed to simulate the intestine. By the term “highly acidic pH” is meant a pH of about 1 to about 4.5. A pH of about 1.2, for example, can be used to simulate the pH of the stomach. By the term “less acidic to basic pH” is meant a pH of greater than about 4 to about 7.5, specifically about 6 to about 7.5. A pH of about 6 to about 7.5, specifically about 6.8, can be used to simulate the pH of the intestine.

The colchicine formulation may be tested using a USP Type I apparatus (basket), at 100 rpm, and 500 mL of dissolution media selected from the group of purified water, acidic buffer of pH 4.5, 0.001 N HCl, 0.1 N HCl, and pH 6.8 phosphate buffer.

An immediate-release colchicine dosage form may exhibit a release profile as measured in an in vitro dissolution test where greater than or equal to about 80% of the active agent is released within two hours, specifically within one hour and more specifically with 30 minutes after combining the formulation with 500 ml of a dissolution medium, e.g. purified water, 0.1 N HCl or 0.001 N HCl. In another embodiment, an immediate-release dosage form may exhibit a release profile as measured in an in vitro dissolution test where not less than about 85% of the active agent is released within 30 minutes after combining the formulation with 500 ml dissolution medium of purified water, 0.1 N HCl or 0.001N HCl. Exemplary dissolution conditions include testing according to USP 34<711>, incorporated herein in its entirety, USP apparatus 1 basket at 37° C.±0.5° C., 100 rpm shaft speed More specifically the dissolution test method, according to the colchicine tablet monograph, is USP apparatus 1, baskets at 37° C.±0.5° C., 100 rpm, 500 ml, purified water with not less than 80 percent of the drug dissolved in 30 minutes (75 Q). Another exemplary dissolution method is USP apparatus 1 baskets at 37° C.±0.5° C., 100 rpm, 500 ml, purified water with not less than 85 percent of the drug dissolved in 30 minutes (80 Q). Yet another method is USP apparatus 2, paddles at 37° C.±0.5° C., 75 rpm, 500 ml purified water, 0.1N HCl, or 0.001N HCl.

In one embodiment, the colchicine formulation exhibits a dissolution profile that is substantially the same as a dissolution profile of an equivalent strength of a reference drug according to New Drug Application No. 022352 wherein the dissolution profile is determined using the conditions according to USP 34<711> test method 1 basket, using of 500 ml of purified water, at 37° C.±0.5° C., and 100 rpm shaft speed; or according to USP 34 <711> test method 2 paddles, using of 500 ml of 0.001N HCl, at 37° C.±0.5° C., and 75 rpm shaft speed. “Substantially the same dissolution profile” means the colchicine formulation releases an amount of active agent within about 10% of the amount released from the reference drug according to New Drug Application No. 022352 (Colcrys®) at any give time point when tested under a dissolution study.

Administration of tablets and capsules can cause patient compliance issues in patients that have difficulty swallowing the dosage forms intact. These patients may include adults (more particularly the elderly), and children. For these patients, an alternative dosage form that is easier to swallow, such as a “sprinkle-able” tablet or capsule formulated in a “sprinkle formulation” that can be sprinkled on food or into a liquid are much more desirable. This is especially critical for patients with swallowing difficulties that rely on frequent or regular medication administration for their general state of health.

Another problem associated with some medications that affects patient compliance is taste. Drug products with bitter or otherwise objectionable taste may be rejected by patients. This may be particularly more problematic for certain medications that are uncoated or otherwise dissolve, degrade, disintegrate or somehow release the active ingredient when the medication is sprinkled on or into a food or liquid administration vehicle.

A sprinkle formulation may not necessarily be administered to a patient immediately after being crushed and/or sprinkled onto an administration vehicle—it may stand for several minutes or longer before the patient receives it. Such a time delay can potentially allow the vehicle, typically applesauce, to disrupt, dissolve, or degrade the sprinkles, exposing the active ingredient to light. This is particularly troublesome with colchicine due to its light sensitivity. Further, a colchicine product that is uncoated or that, after crushing and/or sprinkling on a vehicle, releases the colchicine taste may be rejected by patients. By controlling the particular coating and/or matrix components, photodegradation is minimized which allows the colchicine sprinkle formulations to be sprinkled on applesauce or other food vehicle for several minutes, specifically for up to 5 minutes, more specifically up to 10 minutes, yet more specifically up to 30 minutes, still yet more specifically up to 45 minutes, and more specifically up to 60 minutes or more prior to administration without resulting in the formation of considerable amounts of photodegradants. Further, the stable colchicine sprinkle formulation improves patient compliance by protecting the patient from the bitter taste of the active ingredient. Prevention or reduction of photodegradation results in a stable sprinkle formulation that reduces the potential for toxicity from the photodegradants and permits delayed administration without risking patient health or compliance.

In one embodiment, the colchicine sprinkle formulation is taste masked.

The photostability of the colchicine sprinkle formulation can be analyzed by a photodegradation study to determine whether photodegradants are formed over time when exposed to various types and intensities of light.

Exemplary colchicine photodegradation impurities include beta-Lumicolchicine (N-[(7S,7bR,10aS)-1,2,3,9-tetramethoxy-8-oxo-5,6,7,7b,8,10a-hexahydrobenzo[a]cyclopenta[3,4]cyclobuta[1,2-c]cyclohepten-7-yl]-acetamide) and gamma-Lumicolchicine.

The purity of the colchicine (and therefore the presence of any photodegradants, or ‘impurities’) can be determined using a variety of techniques known in the art such as high pressure liquid chromatography (HPLC), and the like.

The quantitative amount of an individual impurity or of total impurities in colchicine caused by photodegradation may be determined by any suitable analytical method known in the art. In one embodiment, the impurity amounts are determined using a high performance liquid chromatography (HPLC) assay, for example, the HPLC method described in the Colchicine Official Monograph USP30/NF25, herein fully incorporated by reference; Liquid Chromatograph coupled with a Mass Spectrometer, LC-MS; Ultra Performance Liquid Chromatography (UPLC); and the like. Techniques to determine levels of impurities in colchicine include those disclosed in U.S. Patent Application Publication 20090093548 to Davis et al., the contents of which are incorporated herein by reference.

The dosage forms are stable (i.e. result in minimal photodegradation, specifically the formation of not more than 0.06% total beta-lumicolchicine and gamma-lumicolchicine) when exposed to illumination levels from about 400 to about 5000 lux, specifically about 500 to about 3000 lux, more specifically about 1000 to about 2500 lux, and yet more specifically about 1500 to about 2000 lux for a time period of 5 minutes or greater, specifically 10 minutes or greater, more specifically 15 minutes or greater, yet more specifically 30 minutes or greater, and still yet more specifically 45 minutes or greater.

Conversion of lux to footcandles: 1 lux=0.0929 footcandle and 1 footcandle=10.76 lux.

The illumination level is the rate of light energy emission falling on an area as measured by a photometer with an illuminance sensor in lux or foot-candles and indicates brightness. A lux is a unit of illuminance, measured in lumens per square meter. A foot-candle (fc) is lumens per square foot, and is also commonly measured by light meters. The term candela replaced foot-candle as the International System (SI) measure of luminous intensity, and represents one lumen per steradian ° m/s°.

Illumination levels vary depending on lighting conditions, as illustrated in “Implementing Light Aware UI by Using the Windows Sensor and Location Platform” Aug. 23, 2010 http://www.microsoft.com/whdc/device/sensors/light-aware-ui.mspx. As shown, indoor lighting conditions may vary from 400 lux to as high as 5,000 lux. Importantly, medications are typically ingested by individuals or administered to patients in controlled healthcare settings indoors. These medication dosage forms may be subjected to bright illumination conditions as a result of varying lighting in different settings.

In several embodiments, the illumination level comprises between 400 and 5,000 lux.

In several embodiments, the illumination level comprises the recommended United States Pharmacopeia (USP) lighting conditions according to Chapter <1066> Physical Environments That Promote Safe Medication Use Illumination Level. This chapter describes optimal physical environment guidelines that promote accurate medication use and improve the performance of persons involved in the medication use process (e.g., procurement, prescribing, transcribing, order entry, preparation, dispensing, and administration of medications) in any practice setting. This chapter focuses on the characteristics of the physical environment that can promote accurate medication use.

USP recommended illumination levels for healthcare settings include those found in the table below:

| | Illumination Level | |
|---|---|---|
| | | Foot-Candle |
| Work Area | Lux | (fc) |
| Computer order entry | 1000 | 100 |
| Handwritten order processing | 1000 | 100 |
| Medication filling and checking (pharmacy) | 900-1500 | 90-150 |
| Patient counseling (pharmacy) | 900-1500 | 90-150 |
| Sterile compounding and preparation | 1000-1500 | 100-150 |
| Pharmacy medication storeroom | 500 | 50 |
| Medication preparation area, e.g., nursing | 1000 | 100 |
| station | | |
| Medication administration work area (e.g., cart | 1000 | 100 |
| surface) | | |

Exemplary lamps which provide the USP recommended lighting levels include fluorescent cool white deluxe lamps or compact fluorescent lamps.

Recommended USP illumination levels include 500 to 1500 lux, specifically 900 to 1500 lux.

The lamp used in a photodegradation study can also be characterized by its correlated color temperature. The correlated color temperature is the apparent color of the lamp relative to the color appearance of a reference source, typically an incandescent light source, and is measured in degrees Kelvin (K). The correlated color temperature can be characterized according to the manufacturer's specification (e.g. 2700K, 6500K, etc.). In another embodiment, the correlated color temperature can be measured by first operating the lamp continuously for 100 hours and then measuring the lamp output using a spectroradiometer, which has been calibrated (e.g., to NIST standards). In another embodiment, the correlated color temperature of a lamp can be about 2500K to about 7000K, specifically about 2700K to about 6500K. In one embodiment the correlated color temperature is about 2500 to about 2900K, more specifically about 2600 to about 2800K. In another embodiment the correlated color temperature is about 6000 to about 7000K, more specifically about 6200K to about 6600K. In yet another embodiment, the correlated color temperature of the lamp is about 2763K or about 6081K.

Exemplary lamps for use in the photodegradation study include an incandescent lamp, a fluorescent lamp, a compact fluorescent lamp, a halogen lamp, sunlight, and the like.

Also included herein are pharmaceutical products (kits) useful, for example, for the treatment or prevention of gout flares, which comprise one or more containers containing a colchicine formulation as disclosed herein, and optionally information or published material, e.g as product inserts or product labels. The information can indicate quantities of the components to be administered, guidelines for administration, safety issues, and the like.

The kits may further comprise one or more conventional pharmaceutical kit components, such as, for example, one or more containers to aid in facilitating compliance with a particular dosage regimen; one or more carriers; etc. Exemplary kits can be in the form of bubble or blister pack cards, optionally arranged in a desired order for a particular dosing regimen. Suitable blister packs that can be arranged in a variety of configurations to accommodate a particular dosing regimen are well known in the art or easily ascertained by one of ordinary skill in the art.

Also disclosed are methods of treating a patient in need of colchicine therapy by the administration of the colchicine formulation to a patient in need thereof. The colchicine formulations can be used in prevention or treatment of various diseases or conditions, including, for example, attacks of acute gouty arthritis (“gout flares”) and pain in attacks of acute gouty arthritis, prophylaxis of gout flares (often called “chronic gout treatment”), acute pericarditis, asthma, Behçet's disease, cancer, chronic gout (prophylaxis), pseudogout cystic disease comprising polycystic kidney disease or cystic fibrosis, demyelinating disease of central or peripheral origin, Dupuytren's contracture, Familial Mediterranean fever, glaucoma, idiopathic pulmonary fibrosis, idiopathic thrombocytopenic purpura, inflammatory disorder comprising rheumatoid arthritis, lentiviral infection, multiple sclerosis, postpericardiotomy syndrome, primary amyloidosis, primary biliary cirrhosis, proliferative vitreoretinopathy, pyoderma gangrenosum, recurrent pericarditis, or a condition in need of enhanced bone formation or bone mineral density.

In one embodiment, a method for the prophylaxis or treatment of gout flares in adults comprises administering the colchicine formulation to the adult in need of colchicine therapy.

In another embodiment, a method for the treatment of Familial Mediterranean fever (FMF) in adults and children 2 years or older comprises administering the colchicine formulation to the adult or children in need of colchicine therapy.

In one embodiment, the colchicine formulation comprises about 0.1 to about 0.8 mg colchicine per unit dosage form (e.g. per crushable tablet), specifically about 0.2 to about 0.7, more specifically about 0.3 to about 0.65, and still more specifically about 0.5 to about 0.6 mg colchicine per unit dosage form. In one embodiment, the colchicine formulation contains about 0.6 mg colchicine. In another embodiment, the colchicine formulation contains about 0.3 mg colchicine.

In one embodiment, the colchicine formulation is administered for the prophylaxis of gout flares for adults and adolescents older than 16 years of age wherein 0.6 mg of colchicine is administered once or twice daily. A maximum dose for the prophylaxis of gout flares is 1.2 mg/day.

In another embodiment, the colchicine formulation is administered for the treatment of gout flares wherein 1.2 mg of colchicine is administered at the first sign of the flare followed by 0.6 mg one hour later. A maximum recommended dose for treatment of gout flares is about 1.8 mg over a 1 hour period.

In another embodiment, the colchicine formulation is administered for the treatment of FMF in adults and children older than 12 years wherein 1.2 to 2.4 mg of colchicine is administered daily.

In another embodiment, the colchicine formulation is administered for the treatment of FMF in children 6 to 12 years wherein 0.9 to 1.8 mg of colchicine is administered daily, as a single or divided dose twice daily.

In another embodiment, the colchicine formulation is administered for the treatment of FMF in children 2 to 6 years wherein 0.3 to 1.8 mg of colchicine is administered daily, as a single or divided dose twice daily.

In one embodiment, a multiparticulate colchicine capsule formulation comprises a plurality of coated subunits and a capsule; wherein each coated subunit comprises a core subunit and a coating surrounding the core subunit, wherein the core subunit comprises colchicine and a pharmaceutically acceptable excipient, wherein the coating comprises a light blocking material, a light absorbing material, or a light blocking material and a light absorbing material; wherein the capsule can be opened to form a sprinkle formulation comprising the plurality of the coated subunits. Within this embodiment, the sprinkle formulation does not contain more than 0.06% beta-lumicolchicine and gamma-lumicolchicine combined weight after exposure to 1500 lux for 45 minutes (2700K or 6500K).

In one embodiment, a colchicine dosage form comprises colchicine, a pharmaceutically acceptable excipient, and a light protecting agent selected from a light blocking material, a light absorbing material, or a light blocking material and a light absorbing material; wherein the dosage form can be administered as a sprinkle formulation, wherein the sprinkle formulation does not contain more than 0.06% beta-lumicolchicine and gamma-lumicolchicine combined weight after exposure to 1500 to 3000 lux for 15 minutes (about 2700K or about 6500K color temperature). Further within this embodiment, a) the dosage form is a scored tablet; b) the dosage form and its sprinkle form are taste-masked; c) the dosage form has substantially no food effect; d) the dosage form is bioequivalent to Colcrys®; e) the dosage form is a crushable tablet that can be crushed with a low force (e.g., crushable or broken up with finger pressure such as crushable between finger and thumb with minimal effort); f) the dosage form is a scored tablet and the dosage form and its sprinkle form are taste-masked; g) the dosage form has substantially no food effect and the dosage form and its sprinkle form are taste-masked; h) the dosage form is a scored tablet, the dosage form and its sprinkle form is taste-masked, and the dosage form has substantially no food effect; i) the dosage form is a scored, crushable tablet, the dosage form and its sprinkle form are taste-masked, the dosage form has substantially no food effect, and the dosage form is bioequivalent to Colcrys®; or j) any combination thereof. The scored tablet may meet the FDA guidelines and criteria as described herein.

In another embodiment, a multiparticulate colchicine dosage form comprises a plurality of coated subunits; wherein each coated subunit comprises a core subunit and a coating surrounding the core subunit, wherein the core subunit comprises colchicine and a pharmaceutically acceptable excipient, wherein the coating comprises a light blocking material, a light absorbing material, or a light blocking material and a light absorbing material; and wherein the dosage form can be administered as a sprinkle formulation, wherein the sprinkle formulation does not contain more than 0.06% beta-lumicolchicine and gamma-lumicolchicine combined weight after exposure to 1500 to 3000 lux for 15 minutes (about 2700K or about 6500K color temperature). Further within this embodiment, a) the dosage form is a scored tablet; b) the dosage form and its sprinkle form are taste-masked; c) the dosage form has substantially no food effect; d) the dosage form is bioequivalent to Colcrys®; e) the dosage form is a crushable tablet that can be crushed with a low force (e.g., crushable or broken up with finger pressure such as crushable between finger and thumb with minimal effort); f) the dosage form is a scored tablet and the dosage form and its sprinkle form are taste-masked; g) the dosage form has substantially no food effect and the dosage form and its sprinkle form are taste-masked; h) the dosage form is a scored tablet, the dosage form and its sprinkle form is taste-masked, and the dosage form has substantially no food effect; i) the dosage form is a scored, crushable tablet, the dosage form and its sprinkle form are taste-masked, the dosage form has substantially no food effect, and the dosage form is bioequivalent to Colcrys®; or j) any combination thereof. The scored tablet may meet the FDA guidelines and criteria as described herein.

In an embodiment, a colchicine dosage form comprises colchicine, a pharmaceutically acceptable excipient, and a light protecting agent selected from a light blocking material, a light absorbing material, or a light blocking material and a light absorbing material; wherein the dosage form can optionally be administered as a sprinkle formulation, and wherein the dosage form is a tablet comprising two core tablets. In a further embodiment, the dosage form can be split into portions, optionally with the aid of a tablet score, such that each portion comprises a single core tablet with a substantially similar amount of active in each portion. In one embodiment, the colchicine is present in the core tablets and not present in the exterior tablet matrix material. In one embodiment, the core tablets are a monolithic matrix formulation comprising colchicine, a pharmaceutically acceptable excipient, and an optional light protecting agent selected from a light blocking material, a light absorbing material, or a light blocking material and a light absorbing material. Within this embodiment, the core tablets may optionally be coated with a coating comprising a light blocking material, a light absorbing material, or a light blocking material and a light absorbing material. In another embodiment, the core tablets comprise coated subunits as described herein, and a core tablet matrix material comprising a pharmaceutically acceptable material and an optional light protecting agent selected from a light blocking material, a light absorbing material, or a light blocking material and a light absorbing material. Each coated subunit can comprise a core subunit and a coating surrounding the core subunit, wherein the core subunit comprises colchicine and a pharmaceutically acceptable excipient, wherein the coating comprises a light blocking material, a light absorbing material, or a light blocking material and a light absorbing material. Within the forgoing embodiments, the colchicine dosage formulation split in half does not contain more than 0.06% beta-lumicolchicine and gamma-lumicolchicine combined weight after exposure to 1500 to 3000 lux for 15 minutes (about 2700K or about 6500K color temperature). Within the forgoing embodiments, the colchicine dosage form is finger crushable to result in a sprinkle formulation, wherein the sprinkle formulation does not contain more than 0.06% beta-lumicolchicine and gamma-lumicolchicine combined weight after exposure to 1500 to 3000 lux for 15 minutes (about 2700K or about 6500K color temperature).

The following examples further illustrate the invention but, of course, should not be construed as in any way limiting its scope.

### EXAMPLES

### Example 1

### Crushable Tablets Containing Coated Colchicine Granules

Core granules containing colchicine and the components listed in Table 1 are prepared by the process described in U.S. Pat. No. 7,207,505B2 to Bender et al. The resulting core granules had an average diameter of about 74 to about 2000 micrometers.

TABLE 1

| | 1A | 1B |
|---|---|---|
| Core granule | Amount per final | Amount per final |
| components | Tablet (mg) | Tablet (mg) |
| Colchicine | 0.6 | 0.3 |
| Microcrystalline | 20 | 20 |
| Cellulose | | |
| Hypromellose | 10 | 10 |
| Croscarmellose Sodium | 10 | 10 |
| Mannitol\hat{ } | 9.4 | 9.7 |
| Xylitol | 50 | 50 |
| Purified Water | * | * |
| Total Weight Blend | 100 | 100 |
| (mg) | | |

*Does not appear in the final product
\hat{ }amount adjusted based on the potency of the colchicine

The core granules prepared above were coated with one of the following coating compositions listed in Table 2.

TABLE 2

| | 2A. | 2B. | 2C. | 2D. |
|---|---|---|---|---|
| | Uncolored | Purple | Red | Red |
| Coated granule | coating | coating | coating | coating |
| components | Amount per final Tablet (mg) | | | |
| Core granules (1A) | 100 | 100 | 100 | — |
| Core granules (1B) | — | — | — | 100 |
| Ethylcellulose (applied as | 6 | 6 | 6 | 6 |
| an aqueous dispersion of | | | | |
| ethylcellulose, plasticizer | | | | |
| and stabilizer) | | | | |
| Hydroxypropyl | 6 | — | — | — |
| methylcellulose-based | | | | |
| coating material | | | | |
| Purple film coating: | — | 6 | — | — |
| hydroxypropyl | | | | |
| methylcellulose-based | | | | |
| coating material | | | | |
| containing, FD&C Blue | | | | |
| No. 2 aluminum lake, | | | | |
| FD&C Red No. 40 | | | | |
| aluminum lake, titanium | | | | |
| dioxide, and plasticizer | | | | |
| Red film coating: | — | — | 6 | 6 |
| hydroxypropyl | | | | |
| methylcellulose based | | | | |
| coating material | | | | |
| containing, FD&C Red No. | | | | |
| 40 aluminum lake, FD&C | | | | |
| Yellow #6 aluminum lake, | | | | |
| FD&C Blue No. 2 | | | | |
| aluminum lake, titanium | | | | |
| dioxide, and plasticizer | | | | |
| Purified Water | * | * | * | * |
| Total (mg) | 112 | 112 | 112 | 112 |

The coated granules are prepared into crushable tablets containing the components listed in Tables 3a and 3b, 0.6 mg colchicine per final tablet, except for Formulations 9, 10, 17 and 19 which contain 0.3 mg per final tablet. Lactose, starch, mannitol, crosslinked polyvinylpyrrolidone, titanium dioxide (if used) and color (if used) are screened and then mixed together. The coated granules are then blended with the mixture and then screened magnesium stearate is added and blended to form a compressible mixture. The compressible mixture is compressed using tooling 9/32″ SC, except for Formulation 18 which was compressed using a bisected tooling to form a scored tablet.

TABLE 3a

| Crushable tablet | Formulation, Amounts in mg per final tablet | | | | | | | |
|---|---|---|---|---|---|---|---|---|
| component | 1 | 2 | 3 | 4 | 5 | 6 | 7 | 8 |
| Lactose | 11.475 | 11.475 | 9.775 | 10.115 | 10.472 | 10.472 | 9.418 | 9.418 |
| monohydrate* | | | | | | | | |
| Maize starch* | 2.025 | 2.025 | 1.725 | 1.785 | 1.848 | 1.848 | 1.662 | 1.662 |
| Granulated mannitol, | 10 | 10 | 10 | 10 | 10 | 10 | 10 | 10 |
| mean diameter: | | | | | | | | |
| 180 micrometer | | | | | | | | |
| Polyvinylpyrrolidone, | 5 | 5 | 5 | 5 | 5 | 5 | 5 | 5 |
| crosslinked (PVP- | | | | | | | | |
| XL) | | | | | | | | |
| Titanium Dioxide | — | 5.18 | — | 5.18 | 5.18 | 5.18 | — | — |
| Purple Lake Blend A | — | 0.42 | — | — | — | — | 0.42 | — |
| FD&C Blue No. 2 | | | | | | | | |
| aluminum lake, | | | | | | | | |
| FD&C Red No. 40 | | | | | | | | |
| aluminum lake | | | | | | | | |
| Purple Lake Blend B | — | — | — | 0.42 | — | — | — | 0.42 |
| Coated Granules 2A | 111 | 111 | — | — | — | 113 | 113 | — |
| Coated Granules 2B | — | — | 113 | 113 | 113 | — | — | 113 |
| Magnesium stearate | 0.50 | 0.50 | 0.50 | 0.50 | 0.50 | 0.50 | 0.50 | 0.50 |
| Total tablet weight | 140 | 145.6 | 140 | 146 | 146 | 146 | 140 | 140 |

*Spray-dried combination.

TABLE 3b

| Crushable tablet | Formulation, Amounts in mg per final tablet | | | | | | | | | | | |
|---|---|---|---|---|---|---|---|---|---|---|---|---|
| component | 9, 0.3 | 10, 0.3 | 11 | 12 | 13 | 14 | 15 | 16 | 17, 0.3 | 18 | 19, 0.3 | 20 |
| Lactose | 9.401 | 9.401 | 9.401 | 19.125 | 14.365 | 14.518 | 14.722 | 9.401 | 9.401 | 9.401 | 9.401 | 9.401 |
| monohydrate* | | | | | | | | | | | | |
| Maize starch* | 1.659 | 1.659 | 1.659 | 3.375 | 2.535 | 2.562 | 2.598 | 1.659 | 1.659 | 1.659 | 1.659 | 1.659 |
| Granulated mannitol, | 10 | 10 | 10 | 10 | 10 | 10 | 10 | 10 | 10 | 10 | 10 | 10 |
| mean diameter: | | | | | | | | | | | | |
| 180 micrometer | | | | | | | | | | | | |
| Polyvinylpyrrolidone, | 5 | 5 | 5 | 5 | 5 | 5 | 5 | 5 | 5 | 5 | 5 | 5 |
| crosslinked (PVP-XL) | | | | | | | | | | | | |
| Titanium Dioxide | 5.18 | 5.18 | 5.18 | — | 5.18 | — | 5.18 | 5.18 | 5.18 | 5.18 | 5.18 | 5.18 |
| FD&C Red #40 | 0.42 | 0.42 | 1.26 | — | 0.42 | 0.42 | — | — | — | 1.26 | 1.26 | 0.42 |
| Aluminum Lake | | | | | | | | | | | | |
| D&C Yellow #10 | 0.84 | 0.84 | — | — | — | — | — | — | — | — | — | 0.84 |
| Aluminum Lake | | | | | | | | | | | | |
| Lakes | | | | | | | | | | | | |
| Coated Granules 2C | — | — | 112 | 107 | 107 | 112 | 107 | 112 | — | 112 | — | 112 |
| Coated Granules 2D | 112 | 112 | — | — | — | — | — | — | 112 | — | 112 | — |
| Magnesium stearate | 0.50 | 0.50 | 0.50 | 0.50 | 0.50 | 0.50 | 0.50 | 0.50 | 0.50 | 0.50 | 0.50 | 0.50 |
| Total tablet weight | 145 | 145 | 145 | 145 | 145 | 145 | 145 | 143.74 | 143.74 | 145 | 145 | 145 |

*Spray-dried combination.

### Example 2

### Photostability Study

The crushable tablet Formulations 1-8 of Example 1, Table 3a, were studied for photostability by crushing the tablet into a sprinkle formulation and exposing the sprinkle to 1500 lux and 3000 lux light. A Lux Meter made by EXTECH Instruments Corporation, Model Number 407026, Serial Number Z203667, and Light Bulbs made by TCP (2700K catalog number: TCP 8010193 (actual color temperature 2763 K) and 6500K catalog number: TCP 4892365K (actual color temperature 6081K) are used in the study. The color temperature of the light bulbs was determined by first operating the bulbs continuously for 100 hours. The lamp output was measured using a Photo Research PR-735 spectroradiometer, which had been calibrated at the factory to NIST standards. The aperture was set at 1 degree and the radiometric mode was set to irradiance. For testing, the sensor of the PR-735 was positioned 70 inches (177.8 cm) from the emitting surface of each lamp under test. The PR-735 measured the spectral irradiance in W/m² from 380 nm to 1080 nm in 2 nm increments, and this data was used to calculate photometric data such as the correlated color temperature, illuminance and color rendering index using SpectraWin™ 2 software. The bulb TCP 8010193 had an illuminance at 70 inches of 35.83 lux and a correlated color temperature of 2763K and the bulb TCP 4892365K had an illuminance at 70 inches of 47.38 lux and a correlated color temperature of 6081K.

The tablets were crushed by breaking the tablet in half and then crushing the two halves between two fingers. The crushed tablets resulted in a mix of granular and powder particulates where the particulates have a size ranging from about 45 micrometers to about 2000 micrometers, and where about 90% of the particulates have a size of 850 micrometers or less. The sprinkle formulation was then exposed to 1500 lux (lamp manufacturer specified color temperature 2700K and 6500K, actual color temperature 2763K and 6081K) or 3000 lux (lamp manufacturer specified color temperature 2700K and 6500K, actual color temperature 2763K and 6081K) light. Samples at time points 5, 10, 15, 30, 45, 60, 90, 120, 180, and 240 minutes were taken and tested for the percent of beta-lumicolchicine, percent of gamma-lumicolchicine, and percent of the total of the two impurities using Ultra Performance Liquid Chromatography, UV detector, and a mobile phase of ammonium acetate buffer and methanol.

As a comparative example, Colcrys® tablets were crushed by placing the tablet in a mortar and tapping the tablet with a pestle, and then the irregular sized particles are exposed to light in the same manner as discussed above. The results of the photostability study are provided in Table 4 below. “Last time point at which Sample remains stable (minutes)” means the last point where the combined amounts of beta- and gamma-lumicolchicine is not more than 0.06%.

TABLE 4

| | Last time point at which Sample remains stable | | | |
|---|---|---|---|---|
| | in minutes (total % of beta-lumicolchicine and | | | |
| | % gamma-lumicolchicine) | | | |
| | 1500 lux | | 3000 lux | |
| Formulation | 2700K | 6500K | 2700K | 6500K |
| Colcrys ® | 15 | 30 | 5 | 15 |
| crushed tablet | (0.052) | (00.053) | (0.039) | (0.051) |
| 4, 0.6 mg | 45 | 45 | 15 | 30 |
| + color coat | (0.055) | (0.042) | (0.060) | (0.050) |
| + color matrix | | | | |
| + TiO₂ matrix | | | | |
| 5, 0.6 mg | 30 | 45 | 15 | 5 |
| + color coat | (0.055) | (0.058) | (0.056) | (0.012) |
| − color matrix | | | | |
| + TiO₂ matrix | | | | |
| 8, 0.6 mg | 10 | 15 | 5 | 10 |
| + color coat | (0.051) | (0.030) | (0.042) | (0.032) |
| + color matrix | | | | |
| − TiO₂ matrix | | | | |
| 3, 0.6 mg | 10 | 15 | 5 | 10 |
| + color coat | (0.036) | (0.061) | (0.050) | (0.058) |
| − color matrix | | | | |
| − TiO₂ matrix | | | | |
| 2, 0.6 mg | 10 | 5 | 5 | 5 |
| − color coat | (0.046) | (0.038) | (0.057) | (0.040) |
| + color matrix | | | | |
| + TiO₂ matrix | | | | |
| 6, 0.6 mg | 5 | Not stable | Not stable | Not stable |
| − color coat | (0.051) | | | |
| − color matrix | | | | |
| + TiO₂ matrix | | | | |
| 7, 0.6 mg | Not stable | 5 | Not stable | 5 |
| − color coat | | (0.045) | | (0.061) |
| + color matrix | | | | |
| − TiO₂ matrix | | | | |
| 1, 0.6 mg | 5 | 5 | Not stable | Not stable |
| − color coat | (0.061) | (0.062) | | |
| − color matrix | | | | |
| − TiO₂ matrix | | | | |

+ presence of element
− absence of element

As shown by the data, the sprinkle formulations which contained purple coated granules (Formulations 4, 5, 8, and 3) formed lower amounts of beta- and gamma-lumicolchicine compared to the sprinkle formulations containing clear coated granules (Formulations 2, 6, 7, and 1). Surprisingly, the sprinkle formulation containing purple coated granules and no dye or opacifier in the tablet matrix (Formulation 3) performed substantially as well as the sprinkle formulation containing purple coated granules and purple color in the tablet matrix (Formulation 8). Improvement in photostability is achieved in the sprinkle formulation containing purple coated granules and titanium dioxide in the tablet matrix (Formulation 5). However, significant improvement in photostability is achieved in the sprinkle formulation containing both purple color and titanium dioxide in the tablet matrix (Formulation 4), which is surprising since the purple color in the tablet matrix alone (Formulation 8) performed similarly to the corresponding formulation without purple matrix color (Formulation 3).

The crushable tablet Formulations 12, 13, 14, and 15 of Example 1, Table 3b, were studied for photostability by hand-crushing the tablet into a sprinkle formulation and exposing the sprinkle to 3000 lux.

A Lux Meter made by EXTECH Instruments Corporation, Model Number 407026, Serial Number Z203667, and Light Bulbs made by TCP (2700 K catalog number: TCP 8010193 (actual temperature 2763K) and 6500 K catalog number: TCP 4892365K (actual temperature 6081K)) are used in the study. The sprinkle formulation is then exposed to 3000 lux (lamp manufacturer specified color temperature 2700 K and 6500K, actual color temperature 2763K and 6081K) light. Samples at time points 5, 10, 15, 30, 45, 60, 90, 120, 180, and 240 minutes were taken and tested for the percent of beta-lumicolchicine, percent of gamma-lumicolchicine, and percent of the total of the two impurities using Ultra Performance Liquid Chromatography, UV detector, and a mobile phase of ammonium acetate buffer and methanol.

The results of the photostability study are provided in Table 5. below. “Last time point at which Sample remains stable (minutes)” means the last point where the combined amounts of beta- and gamma-lumicolchicine is not more than 0.06%.

TABLE 5

| | Last time point at which | | Last time point at which | |
|---|---|---|---|---|
| | Sample remains stable in | | Sample is not stable in | |
| | minutes (total % beta- | | minutes (total % beta- | |
| | lumicolchicine and % | | lumicolchicine and % | |
| | gamma-lumicolchicine) | | gamma-lumicolchicine) | |
| | 3000 Lux | | 3000 lux | |
| Formulation | 2700K | 6500K | 2700K | 6500K |
| 12, 0.6 mg | 5 | 5 | 10 | 10 |
| + color coat | (0.04) | (0.03) | (0.11) | (0.07) |
| − color matrix | | | | |
| − TiO₂ matrix | | | | |
| 15, 0.6 mg | 5 | 10 | 10 | 15 |
| + color coat | (0.03) | (0.05) | (0.07) | (0.08) |
| − color matrix | | | | |
| + TiO₂ matrix | | | | |
| 13, 0.6 mg | 10 | 15 | 15 | 30 |
| + color coat | (0.04) | (0.05) | (0.07) | (0.08) |
| + color matrix | | | | |
| + TiO₂ matrix | | | | |
| 14, 0.6 mg | 5 | 15 | 10 | 30 |
| + color coat | (0.04) | (0.05) | (0.07) | (0.13) |
| + color matrix | | | | |
| − TiO₂ matrix | | | | |
| 9, 0.3 mg | 30 | 45 | — | — |
| + color coat | (0.05) | (0.05) | | |
| + color matrix | | | | |
| + TiO₂ matrix | | | | |

+ presence of element
− absence of element

As shown by the data, the sprinkle formulation which contained the combination of red dye and opacifier in the tablet matrix (Formulation 13) exhibited significantly better photostability than a tablet matrix without a light protecting agent (Formulation 12) or even the formulations containing either red dye (Formulation 14) or opacifier (Formulation 15).

### Example 3

### Photostability Study: Sprinkle in Applesauce

Formulations 11, 16, 18, 20 (0.6 mg colchicine), 10, 17, 19 (0.3 mg colchicine) of Table 3b, and Colcrys® (0.6 mg colchicine) were studied for photostability as a sprinkle in applesauce. One tablet of Formulation 11 was broken in half and each half was crushed by hand into granules and sprinkled on top of applesauce (10 ml Mussleman's Natural Unsweetened Apple Sauce). One tablet each of Formulations 10, 16, 17, 19, and 20 were similarly prepared.

One sample of Formulation 18 was prepared as Formulation 11. A second sample of Formulation 18 was split in half, and one half (0.3 mg) was crushed by hand into granules and sprinkled on top of 10 ml applesauce.

A tablet of Colcrys® was crushed into powder fragments of irregular size using a mortar and pestle. The crushed tablet was sprinkled on the same quantity and type of applesauce used for Formulation 11. A separate tablet of Colcrys® was similarly crushed and not combined with applesauce. A third tablet of Colcrys® was prepared into chunks by tapping the tablet once with mortar and pestle to form large and small chunks of uneven sizes, generally less than the size of one-half the tablet, which were exposed to light without combining with applesauce.

Each sample was exposed to 1500 lux (lamp manufacturer specified color temperature 2700 K and 6500K, actual color temperature 2763K and 6081K). Formulations 16 and 17 were also exposed to light without combining with apple sauce. Samples at time points 5, 10, 15, 30, 45, 60, 90, 120, 180, and 240 minutes were taken and tested for the percent of beta-lumicolchicine, percent of gamma-lumicolchicine, and percent of the total of the two impurities using the test method of Example 2.

The results of the photostability study are provided in Table 6. below. “Last time point at which Sample remains stable (minutes)” means the last point where the combined amounts of beta- and gamma-lumicolchicine is not more than 0.06%.

TABLE 6

| | | Last time point at which Sample remains | |
|---|---|---|---|
| | | stable in minutes (total % beta- | |
| | | lumicolchicine and % gamma-lumicolchicine) | |
| | Apple- | 1500 lux | |
| Formulation | sauce | 2700K | 6500K |
| 11, 0.6mg | Yes | 90 | 240 |
| + color coat | | (0.06) | (0.05) |
| + color matrix | | | |
| + TiO₂ matrix | | | |
| Colcrys ® | Yes | 60 | 120 |
| tablet chunks | | (0.04) | (0.04) |
| 10, 0.3 mg | Yes | 45 | 120 |
| + color coat | | (0.06) | (0.05) |
| + color matrix | | | |
| + TiO₂ matrix | | | |
| 16, 0.6 mg | Yes | 30 | 90 |
| + color coat | | (0.05) | (0.04) |
| − color matrix | | | |
| + TiO₂ matrix | | | |
| Colcrys ® | Yes | 30 | 90 |
| powder | | (0.06) | (0.06) |
| 16, 0.6 mg | No | 30 | 60 |
| + color coat | | (0.03) | (0.05) |
| − color matrix | | | |
| + TiO₂ matrix | | | |
| 17, 0.3 mg | Yes | 15 | 90 |
| + color coat | | (0.03) | (0.05) |
| − color matrix | | | |
| + TiO₂ matrix | | | |
| 17, 0.3 mg | No | 15 | 30 |
| + color coat | | (0.06) | (0.06) |
| − color matrix | | | |
| + TiO₂ matrix | | | |
| Colcrys ® | No | 5 | 5 |
| powder | | (0.04) | (0.05) |
| 18, 0.6 mg | Yes | 60 | 120 |
| + color coat | | (0.06) | (0.05) |
| − color matrix | | | |
| + TiO₂ matrix | | | |
| 18, 0.3 mg | Yes | 45 | 120 |
| + color coat | | (0.04) | (0.04) |
| − color matrix | | | |
| + TiO₂ matrix | | | |
| 19, 0.3 mg | Yes | 60 | 120 |
| + color coat | | (0.05) | (0.06) |
| + color matrix | | | |
| + TiO₂ matrix | | | |
| 20, 0.6 mg | Yes | 60 | 180 |
| + color coat | | (0.06) | (0.06) |
| + color matrix | | | |
| + TiO₂ matrix | | | |

+ presence of element
− absence of element

The results show sprinkle Formulation 11 containing red coated granules and red lake/titanium dioxide in the tablet matrix exhibited improved photostability in applesauce compared with Colcrys®. The data also shows the higher dosage strength (0.6 mg) is generally more stable than the lower strength (0.3 mg).

### Example 4

### Photostability Study in Water

Formulation 11 (0.6 mg colchicine) and Colcrys® (0.6 mg colchicine) were studied for photostability in 10 ml water. One tablet of Formulation 11 was broken in half and each half was crushed by hand into granules and sprinkled into 10 ml water. One tablet of Colcrys® was crushed into fragments of irregular size using a pestle and weighing paper. The crushed tablet was sprinkled into the same quantity of water. Each sample was exposed to 1500 lux (lamp manufacturer specified color temperature 2700 K and 6500K, actual color temperature 2763K and 6081K). Samples at time points 5, 10, 15, 30, 45, 60, 90, 120, 180, and 240 minutes were taken and tested for the percent of beta-lumicolchicine, percent of gamma-lumicolchicine, and percent of the total of the two impurities using the test method of Example 2.

The results of the photostability study are provided in Table 7. below. “Last time point at which Sample remains stable (minutes)” means the last point where the combined amounts of beta- and gamma-lumicolchicine is not more than 0.06%.

TABLE 7

Last time point at which Sample remains stable
in minutes (total % beta-lumicolchicine and %
gamma-lumicolchicine)
1500 lux

| Formulation | 2700K | 6500K |
|---|---|---|
| Colcrys ® | 10 | 15 |
| crushed tablet | (0.046) | (0.055) |
| 11, 0.6 mg | 15 | 45 |
| + color coat | (0.031) | (0.037) |
| + color matrix | | |
| + TiO₂ matrix | | |

+ presence of element
− absence of element

The results show sprinkle Formulation 11 containing red coated granules and red lake/titanium dioxide in the tablet matrix exhibited superior photostability in water compared with Colcrys®.

The terms “comprising”, “having”, “including”, and “containing” are to be construed as open-ended terms (i.e., meaning “including, but not limited to”). The terms “a” and “an” do not denote a limitation of quantity, but rather denote the presence of at least one of the referenced item. The term “or” means “and/or”. The endpoints of all ranges directed to the same component or property are inclusive and independently combinable.

Unless defined otherwise, technical and scientific terms used herein have the same meaning as is commonly understood by one of skill in the art to which this invention belongs.

Embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A colchicine dosage form, comprising:
colchicine,
a pharmaceutically acceptable excipient, and
a light protecting agent selected from a light blocking material, a light absorbing material, or a light blocking material and a light absorbing material;
wherein the dosage form can be administered as a sprinkle formulation, wherein the sprinkle formulation does not contain more than 0.06% beta-lumicolchicine and gamma-lumicolchicine combined weight after exposure to 1500 to 3000 lux for 15 minutes (about 2700K or about 6500K color temperature).

2. The dosage form of claim 1, comprising a plurality of subunits comprising colchicine, the pharmaceutically acceptable excipient, and the light protecting agent selected from a light blocking material, a light absorbing material, or a light blocking material and a light absorbing material.

3. The dosage form of claim 1, wherein the light protecting agent is present in the dosage form in one or more of a coating, a matrix excipient, or as a granulating excipient.

4. A multiparticulate colchicine dosage form, comprising:
a plurality of coated subunits;
wherein each coated subunit comprises a core subunit and a coating surrounding the core subunit,
wherein the core subunit comprises colchicine and a pharmaceutically acceptable excipient,
wherein the coating comprises a light blocking material, a light absorbing material, or a light blocking material and a light absorbing material; and
wherein the dosage form can be administered as a sprinkle formulation, wherein the sprinkle formulation does not contain more than 0.06% beta-lumicolchicine and gamma-lumicolchicine combined weight after exposure to 1500 to 3000 lux for 15 minutes (about 2700K or about 6500K color temperature).

5. The dosage form of claim 4, in the form of a crushable tablet comprising the plurality of coated subunits and a tablet matrix;
wherein the tablet matrix comprises a pharmaceutically acceptable excipient, and a light blocking material, a light absorbing material or a light blocking material and a light absorbing material; and
wherein the tablet can be crushed to form the sprinkle formulation.

6. The dosage form of claim 1, wherein the sprinkle formulation does not contain more than 0.06% beta-lumicolchicine and gamma-lumicolchicine combined weight after exposure to 1500 to 3000 lux for 30 minutes (about 2700K or about 6500K color temperature).

7. The dosage form of claim 1, wherein the sprinkle formulation does not contain more than 0.06% beta-lumicolchicine and gamma-lumicolchicine combined weight after exposure to 1500 lux for 45 minutes (about 2700K or about 6500K color temperature).

8. The dosage form of claim 4, wherein the coated subunits or subunits have an average length of its longest dimension of about 20 to about 2000 micrometers.

9. The dosage form of claim 1, wherein the light blocking material is titanium dioxide, an iron oxide, zinc oxide, aluminum oxide, kaolin, calcium carbonate, or a combination thereof.

10. (canceled)

11. The dosage form of claim 1, wherein the light absorbing material is FD&C Red No. 40 Aluminum Lake; FD&C Red No. 4 Lake; D&C Red No. 6 Lake; D&C Red No. 7 Lake; D&C Red No. 17 Lake; D&C Red No. 21 Lake; D&C Red No. 22 Lake; D&C Red No. 27 Lake; D&C Red No. 28 Lake; D&C Red No. 30 Lake; D&C Red No. 31 Lake; D&C Red No. 33 Lake; D&C Red No. 34 Lake; D&C Red No. 36 Lake; D&C Violet No. 2 Lake; D&C Yellow No. 10 Aluminum Lake; FD&C Yellow No. 6 Aluminum Lake; FD&C Yellow No. 5 Lake; D&C Yellow No. 7 Lake; D&C Yellow No. 8 Lake; FD&C Blue No. 1 Lake; FD&C Blue No. 2 Aluminum Lake; D&C Blue No. 4 Lake; FD&C Green No. 3 Lake; D&C Green No. 5 Lake; D&C Green No. 6 Lake; D&C Orange No. 4 Lake; D&C Orange No. 5 Lake; D&C Orange No. 10 Lake; D&C Orange No. 11 Lake; FD&C Red No. 40; FD&C Red No. 4; D&C Red No. 6; D&C Red No. 7; D&C Red No. 17; D&C Red No. 21; D&C Red No. 22; D&C Red No. 27; D&C Red No. 28; D&C Red No. 30; D&C Red No. 31; D&C Red No. 33; D&C Red No. 34; D&C Red No. 36; D&C Red No. 39; D&C Violet No. 2; FD&C Yellow No. 6; FD&C Yellow No. 5; D&C Yellow No. 7; D&C Yellow No. 8; D&C Yellow No. 10; D&C Yellow No. 11; FD&C Blue No. 1; FD&C Blue No. 2; D&C Blue No. 4; D&C Blue No. 9; FD&C Green No. 3; D&C Green No. 5; D&C Green No. 6; D&C Green No. 8; D&C Orange No. 4; D&C Orange No. 5; D&C Orange No. 10; D&C Orange No. 11; or a combination thereof.

12. The dosage form of claim 1, wherein the light absorbing material is FD&C Red No. 40 Aluminum Lake; D&C Yellow No. 10 Aluminum Lake; FD&C Yellow No. 6 Aluminum Lake; FD&C Blue No. 2 Aluminum Lake; or a combination thereof.

13. The dosage form of claim 1, wherein the light absorbing material is a combination of FD&C Red No. 40 aluminum lake and FD&C Blue No. 2 aluminum lake; and wherein the light blocking material is titanium dioxide.

14. The dosage form of claim 1, wherein the light absorbing material is a combination of FD&C Red No. 40 aluminum lake and D&C Yellow No. 10 aluminum lake; and wherein the light blocking material is titanium dioxide.

15. The dosage form of claim 3, wherein the light blocking material of the tablet matrix is present in an amount of about 1 to about 15 wt % based on the total weight of the tablet.

16. The dosage form of claim 3, wherein the light absorbing material of the tablet matrix is present in an amount of about 0.01 to about 1.5 wt % based on the total weight of the tablet.

17. The dosage form of claim 3, wherein the coating further comprises a film forming polymer and a plasticizer;
wherein the film forming polymer is an alkylcellulose; a hydroxyalkylcellulose; a hydroxyalkyl alkylcellulose; a carboxyalkylcellulose; an alkali metal salt of a carboxyalkylcellulose; a carboxyalkyl alkylcellulose; a carboxyalkylcellulose ester; a starch; a pectine; a chitine derivate; alginic acid or an alkali metal or ammonium salt thereof; a carrageenan; a galactomannan; traganth; agar-agar; gum arabicum; guar gum; xanthan gum; a polyacrylic acid or salt thereof; a polymethacrylic acid or a salt thereof; a methacrylate copolymer; a polyvinylalcohol; a polyvinylpyrrolidone; a copolymer of polyvinylpyrrolidone with vinyl acetate; a polyalkylene oxide or a combination thereof.

18. The dosage form of claim 5, wherein the tablet meets one or more of the following properties:
friability of not more than 0.8%; and
tensile strength of about 10 to about 8000 kPascal.

19. The dosage form of claim 1, in the form of a scored, crushable tablet; or in the form of a scored, crushable tablet having a functional score.

20. (canceled)

21. The dosage form of claim 1, wherein the dosage form is taste masked.

22. (canceled)

23. The dosage form of claim 1, wherein the dosage form exhibits a ratio of a geometric mean of logarithmic transformed AUC_{0-INF} of the dosage form to a geometric mean of logarithmic transformed AUC_{0-INF} of reference drug (New Drug Application No. 022352) of about 0.80 to about 1.25;
a ratio of a geometric mean of logarithmic transformed AUC₀₋ₜ of the dosage form to a geometric mean of logarithmic transformed AUC₀₋ₜ of reference drug (New Drug Application No. 022352) of about 0.80 to about 1.25;
a ratio of a geometric mean of logarithmic transformed Cₘₐₓ of the dosage form to a geometric mean of logarithmic transformed Cₘₐₓ of reference drug (New Drug Application No. 022352) of about 0.70 to about 1.43; or
a ratio of a geometric mean of logarithmic transformed Cₘₐₓ of the dosage form to a geometric mean of logarithmic transformed Cₘₐₓ of reference drug (New Drug Application No. 022352) of about 0.80 to about 1.25,
wherein the foregoing are determined under fasting or non-fasting conditions; or
wherein the dosage form is bioequivalent to a reference drug according to New Drug Application No. 022352 when administered to a patient in a fasted or non-fasted state.

24. (canceled)

25. The dosage form of claim 1,
wherein the dosage form when administered to a patient in a non-fasted state is bioequivalent to the dosage form when administered to a patient in a fasted state;
wherein the dosage form exhibits a ratio of a geometric mean of logarithmic transformed AUC_{0-INF} of the dosage form administered in a non-fasted state to a geometric mean of logarithmic transformed AUC_{0-INF} of the dosage form administered in a fasted state of about 0.80 to about 1.25;
wherein the dosage form exhibits a ratio of a geometric mean of logarithmic transformed AUC₀₋ₜ of the dosage form administered in a non-fasted state to a geometric mean of logarithmic transformed AUC₀₋ₜ of the dosage form administered in a fasted state of about 0.80 to about 1.25; or
wherein the dosage form exhibits a ratio of a geometric mean of logarithmic transformed Cₘₐₓ of the dosage form administered in a non-fasted state to a geometric mean of logarithmic transformed geometric mean Cₘₐₓ of the dosage form administered in a fasted state of about 0.80 to about 1.25.

26. The dosage form of claim 1, wherein the dosage form has less than a 20% variation for AUC_{0-INF}, AUC₀₋ₜ, Cₘₐₓ, or a combination thereof between a fasting state and a non-fasting state.

27. A method of treating a patient in need of colchicine therapy, comprising:
administering to a patient in need thereof the dosage form of claim 1.

28. The method of claim 27, wherein the dosage form is administered for prevention or treatment of attacks of acute gouty arthritis and pain in attacks of acute gouty arthritis, prophylaxis of gout flares, acute pericarditis, asthma, Behçet's disease, cancer, chronic gout (prophylaxis), pseudogout cystic disease comprising polycystic kidney disease or cystic fibrosis, demyelinating disease of central or peripheral origin, Dupuytren's contracture, Familial Mediterranean fever, glaucoma, idiopathic pulmonary fibrosis, idiopathic thrombocytopenic purpura, inflammatory disorder comprising rheumatoid arthritis, lentiviral infection, multiple sclerosis, postpericardiotomy syndrome, primary amyloidosis, primary biliary cirrhosis, proliferative vitreoretinopathy, pyoderma gangrenosum, recurrent pericarditis, or a condition in need of enhanced bone formation or bone mineral density.
